SEQUENCE LISTING (18 sequences)

SEQ ID NO: 1 - DNA, 200 bp, Cannabis sativa
  source 1..200: mol_type = genomic DNA
  variation 101: replace = a
cgatgatcaagatacttgctaaggagctraaggggayggggatcacggccaactgcktggcmccgggaccaatcgcgacsgatatgttcttttcggggaagagtgaggatgacttgaagaaagtggtggatgagtgccccatggggaggcttggagaggttaaggatgtggttccwgtggtgggattcttggctggtgat

SEQ ID NO: 2 - DNA, 200 bp, Cannabis sativa
  source 1..200: mol_type = genomic DNA
  variation 101: replace = a
catgcaagtagtggtgtaaatggctttcaactgggaatgattcatataagaactccttgtaatattctttacgaggtgcatggcagaggatcacacatttcccggaattatctagcaatggccgactagcatgacccatcatctgcaacagatccgcgacaggataatctgtgtggacattttcacggccatcatagtat

SEQ ID NO: 3 - DNA, 200 bp, Cannabis sativa
  source 1..200: mol_type = genomic DNA
  variation 101: replace = g
tttgatygattmtttttwcaaacctgtaattaattcttcgagaacaagtaacyaaaaycttctctttgttcctgaaaaacggagttgattcagtttctgsttctgcttcattyttcttctctttccaacmcaaaagwgtcctttkaggcctctctgcagctgtatcatccttcttcactggtgccaccaccacagtctca

SEQ ID NO: 4 - DNA, 200 bp, Cannabis sativa
  source 1..200: mol_type = genomic DNA
  variation 101: replace = t
aawtgcattgttgattattttcaaattttctaattctaaataggtttgtgcatggagatgtaaagcccgagaattttttacttggtcaacctggaacagtcgatgagaagaaactatatcttgttgatcttggaytgggtattgtttcttttccgasttcttatcctttyaaccgtgtcatttttytgaattttctcaca

SEQ ID NO: 5 - DNA, 200 bp, Cannabis sativa
  source 1..200: mol_type = genomic DNA
  variation 101: replace = t
tttttwcaaacctgtaattaattcttcgagaacaagtaacyaaaaycttctctttgttcctgaaaaacggagttgattcagtttctgsktctgcttcattcttcttctctttccaacmcaaaagwgtcctttkaggcctctctgcagctgtatcatccttcttcactggtgccaccaccacagtctcactgtgctttctc

SEQ ID NO: 6 - DNA, 151 bp, Cannabis sativa
  source 1..151: mol_type = genomic DNA
  variation 76: replace = g
tcaaagtccaacackatcgaaagctccactaattcttgactgggttctgytgtagagaggaaacakagccatggctacgtccatkgcctctyaattasaggccatcaawtccttcctacaagctattatctttaaatcyraagctacttaa

SEQ ID NO: 7 - DNA, 44 bp, synthetic construct
  source 1..44: mol_type = other DNA
gaaggtgaccaagttcatgctagagaggaaacakagccatggct

SEQ ID NO: 8 - DNA, 43 bp, synthetic construct
  source 1..43: mol_type = other DNA
gaaggtcggagtcaacggattgagaggaaacakagccatggcg

SEQ ID NO: 9 - DNA, 25 bp, synthetic construct
  source 1..25: mol_type = other DNA
gcttgtaggaaggawttgatggcct

SEQ ID NO: 10 - DNA, 950 bp, Cannabis sativa
  source 1..950: mol_type = genomic DNA
tctgcccattttccttatgttttaatttcaaactggttggtaggtttttattgaagaaaatttaaatgatacagagaggaaaaaatatttcaatctacaacaagtaatatgtgatcttagtggtatgtaagtataatttttttttttttaaaaaaaaagagtatatgtattttatgtaaaataaaaaagataaaataaagaatatttatatatttttgaggtataaataaaattttattaaaatattatttatctcttaaaaatatcatatctttatatattaaaagtggctatgtaacgataatttttggtttaacgattatttttaatggagctttttctaaataaaatagacgaaacattaacgcttcgacgctaagcatctctccttgctcctcgtttgtttgttagtttcaaaatccaaattaatctgttgaattttgcaattttttggtaaagcgatttagtgatgaaacctcttaacaataatgaaataaaccataaaacattttcaaagtccaacacgatcgaaagctccactaattcttgactgggttctgctgtagagaggaaacagagccatggctacgtccatggcctcttaattacaggccatcaaatccttcctacaagctattatctttaaatctgaagctacttaattgatgttgaaactattctctatattgcccttgaaggtcagccactcgctcaatctctcccaattatttctttgggccacgccctcaccgagtaagactaagagatggatataacattactcaaatcatcattgtccccgagagatctcaccttggtattctctttctcttaatctatgtttctctctacacttttttgttattattcactgtaaacattagttttgaaaagaacgaaaattttataataataattattctattataaaaatatattatacgatgccc

SEQ ID NO: 11 - DNA, 1013 bp, Cannabis sativa
  source 1..1013: mol_type = genomic DNA
aatttttttttttcctcctccaaaaacactcgtgaagattcgatcgaactttcaggtcttcactctcaatttttttgcatatatttttgtttatgcacgcaaactgtttgaataaatgtctctttgaaaatctaacaaaagggatcctaaaagtgttataatttcttatgttaatgctcacaaactgtttgaataaatgtctctttgaaaatctaactaaaggggatcctaaaagtgttataatttcttatgttaatgctcacaaactgtttgaataaatgtctctttgaaaatctaactaaagggatcctaaaagtgttataatttcttatgttaatactcacaaagtgtttgaataattgtcagtgtgaatacaagaggagctttcttgtgcctaagagaggcagcaaacaggttgaagcgtggcggcgtgggtaggatcatatcgatatcttcgtccatggtggggggattgaagccgggattcggggtgtatgcagcgtcaaaggcggctgtggagacgatgatcaagatacttgctaaggagctaaaggggacggggatcacggccaactgcgtggccccgggaccaatcgcgaccgatatgttcttttcggggaaaagtgaggatgacttgaagaaagtggtggatgagtgccccatggggaggcttggagaggttaaggatgtggttcctgtggtgggattcttggctggtgatgggagtgaatgggttaatgggcaggtcattcgtgcaaatggggggtatgtttaagatcaagtctcttgaaaagatgccaactttctaagattttgatcgtggttttatcataataataagcattagagttctaaactcattgaaaatgtgaaataaaatcaacggttagtgttagatgatgatgatgatttaagtatatatatgtataatgggaactttgtatttttttttagtcgaaactcaaattgtagttgcatgattttcaatgagaagttaagagaggttgattgcc

SEQ ID NO: 12 - DNA, 323 bp, Cannabis sativa
  source 1..323: mol_type = genomic DNA
tttattaagcgcccaagctcccaccttactttgatcagactcaacctgttatctttaggccaaagaggcacaagtgagacaatattcaacatcaaatgacgatactaataatcataataaaaattagttcatccccaacaaaattcccaaaaagaaaaagcagattaagaaatttgatcgattctttttacaaacctgtaattaattcttcgagaacaagtaaccaaaaccttctctttgttcctgaaaaacggagttgattcagtttctgggtctgcttcatctcttctcttccaacccaaaagatctgagctagtcattag

SEQ ID NO: 13 - DNA, 23 bp, Cannabis sativa
  source 1..23: mol_type = genomic DNA
ccaccaatggttgcaactccaca

SEQ ID NO: 14 - DNA, 24 bp, Cannabis sativa
  source 1..24: mol_type = genomic DNA
tctggtgagatttgatattgatac

SEQ ID NO: 15 - DNA, 84 bp, Cannabis sativa
  source 1..84: mol_type = genomic DNA
gttggtgctttacttactgggttggtttttgtttttgtctttcaagtgtttgcttatatgtcttgtttttttctttttatgcag

SEQ ID NO: 16 - DNA, 55 bp, Cannabis sativa
  source 1..55: mol_type = genomic DNA
agttttttccccatctggtgtttctagcatgcaatggaagacaggagctgaacca

SEQ ID NO: 17 - DNA, 75 bp, Cannabis sativa
  source 1..75: mol_type = genomic DNA
acaagtaagttgttcaacaagctcaatatctttgcttttgctttgtttaaatcttcgaaagatgaattcataagt

SEQ ID NO: 18 - DNA, 88 bp, Cannabis sativa
  source 1..88: mol_type = genomic DNA
gtactctatttattcatgtataataataaaaactccccttttttgttaattccaccaatttgaacttttaaacggcttttctctgcag

(19) United States
(12) Patent Application Publication
(10) Pub. No.: US 2026/0305549 A1
(43) Pub. Date: Oct. 8, 2026

(54) GENETIC MARKERS FOR POWDERY MILDEW RESISTANCE IN CANNABIS AND RELATED METHODS

(71) Applicant: AURORA CANNABIS ENTERPRISES INC., Vancouver (CA)

(72) Inventors: Soren Seifi, Comox (CA); Jose Celedon, Comox (CA); Keegan Leckie, Comox (CA); Ingrid Giles, Comox (CA)

(73) Assignee: AURORA CANNABIS ENTERPRISES INC., Vancouver, BC (CA)

(21) Appl. No.: 19/479,911

(22) PCT Filed: Apr. 30, 2024
(86) PCT No.: PCT/CA2024/050583

§ 371 Date: Oct. 29, 2025

Related U.S. Application Data
(60) Provisional application No. 63/463,266, filed on May 1, 2023.

(51) Int. Cl. A01H 1/04; A01H 1/00; C12Q 1/6858; C12Q 1/6895

(52) U.S. Cl. CPC A01H 1/045; A01H 1/1255; C12Q 1/6858; C12Q 1/6895; C12Q 2600/156

(57) ABSTRACT

Genetic markers for powdery mildew resistance in Cannabis are provided. In some embodiments, the genetic markers may be on chromosome 9 of the Cannabis genome. In some embodiments, the genetic markers may be single nucleotide polymorphisms (SNPs). Related methods for testing Cannabis plants using such markers and methods for producing Cannabis plants with powdery mildew resistance are also provided.

### CROSS REFERENCE TO RELATED APPLICATION

The present disclosure claims priority to U.S. Provisional No. 63/463,266, filed May 1, 2023, the entire content of which is herein incorporated by reference.

### TECHNICAL FIELD

The present disclosure relates to genetic markers for plant traits. More particularly, the present disclosure relates to genetic markers for powdery mildew resistance in Cannabis and related methods.

### BACKGROUND

Cannabis refers to plants of the Cannabis genus. The Cannabis genus is generally understood to comprise one species, Cannabis sativa L., although some botanical authorities also recognize Cannabis indica and Cannabis ruderalis. A variety of economically important products can be derived from Cannabis plants including seed oil, fiber and medicinal and psychoactive secondary metabolites (known as cannabinoids) such as cannabidiolic acid (CBDA) and tetrahydrocannabinolic acid (THCA).

Powdery mildew (PM) is one of the most widely spread and damaging diseases of many indoor, greenhouse and field grown crops around the world including Cannabis cultivations. PM disease in Cannabis is commonly caused by two species of the biotrophic fungal pathogen Golovinomyces, namely G. ambrosiae and G. cichoracearum (Pépin et al., 2018; Scott and Punja, 2021). PM infection attacks the leaves, stems and flowers of cannabis, causing premature leaf drop, poor flower quality and significant yield losses (Mihalyov and Garfinkel, 2021; Scott and Punja, 2021). The infection cycle in PM has three main phases: (1) spore germination; (2) mycelial network development; and (3) spore (conidia) generation (also known as sporulation) (Hückelhoven, 2005). PM can often complete its life cycle within 1-2 weeks post inoculation (wpi) on susceptible Cannabis genotypes.

Application of chemical fungicides and biological agents, performing strict cultural practices, and growing genetically resistant cultivars have been historically used to manage PM diseases on other agricultural crops, with the latter typically providing the most sustainable, effective, and economical outcome (AGRIOS, 2005). With respect to Cannabis, agrochemicals and biological products are tightly controlled by regulatory agencies in legal Cannabis markets throughout the world, and the few available products often exhibit limited protection (Mihalyov and Garfinkel, 2021; Scott and Punja, 2021). Hence, the development of commercial cultivars with genetic resistance to PM remains highly valuable and sought after goal in the Cannabis industry.

In Cannabis, breeding for PM resistance has been considerably impeded by the lack of access to naturally evolved genetic sources of resistance. Conventional breeding activities in the legacy market during the past decades have been mainly focused on higher potency and yield. This trend, coupled with the liberal use of pesticides to control pest and pathogens, has resulted in the loss of disease resistance (R) genes in the genetic pool of commercially available Cannabis cultivars. Recently, Mihalyov and Garfinkel (2021) reported the discovery of a PM disease R gene in Cannabis located on chromosome 2 (Chr ID: NC_044375.1; GenBank acc. no. GCA_900626175.2) that the authors designated PM1. However, PM1 does not appear to confer complete resistance to PM and other R genes for PM in Cannabis have yet to be identified. As such, identification and selective breeding of Cannabis cultivars with resistance to PM remains a challenge.

### SUMMARY

In one aspect, there is provided a method comprising: providing nucleic acid from a Cannabis plant; and testing the nucleic acid to determine the presence or absence of a variant at a polymorphic site on chromosome 9 of the Cannabis genome, wherein the presence of the variant in one or both copies of the polymorphic site indicates that the plant has a powdery mildew resistant phenotype.

In some embodiments, the polymorphic site is located within a region on chromosome 9 of the Pink Pepper reference genome selected from: between about 57 megabases to about 60 megabases, between about 57.4 to about 59.5 megabases, between about 58 to about 59 megabases, between about 58.1 to about 58.6 megabases, or between about 58,164,118 and about 58,552,299 bases.

In some embodiments, the polymorphic site is located within a region on chromosome 9 of the CBDRx reference genome selected from: between about 56 megabases to about 60 megabases, between about 56.2 to about 59.2 megabases, between about 58 to about 59 megabases, between about 58.1 to about 58.6 megabases, or between about 58,108,801 to about 58,570,898 bases.

In some embodiments, the polymorphic site is selected from: position 101 of SEQ ID NO: 1 or its complement; position 101 of SEQ ID NO: 2 or its complement; position 101 of SEQ ID NO: 3 or its complement; position 101 of SEQ ID NO: 4 or its complement; position 101 of SEQ ID NO: 5 or its complement; or another polymorphic site in linkage disequilibrium therewith.

In some embodiments, the polymorphic site is position 101 of SEQ ID NO: 1 or its complement, and wherein the variant is an A or a T in the complement; the polymorphic site is position 101 of SEQ ID NO: 2 or its complement, and wherein the variant is an A or a T in the complement; the polymorphic site is position 101 of SEQ ID NO: 3 or its complement, and wherein the variant is a G or a C in the complement; the polymorphic site is position 101 of SEQ ID NO: 4 or its complement, and wherein the variant is a T or an A in the complement; or the polymorphic site is position 101 of SEQ ID NO: 5 or its complement, and wherein the variant is a T or an A in the complement.

In some embodiments, the polymorphic site is one of the polymorphic sites in Table 3 or another polymorphic site in linkage disequilibrium therewith.

In some embodiments, the testing comprises nucleic acid amplification.

In some embodiments, the testing is performed using an allele specific method.

In some embodiments, the testing is performed using allele-specification amplification, allele-specific probe hybridization, allele-specific primer extension, sequencing, 5′ nuclease digestion, molecular beacon assay, oligonucleotide ligation assay, size analysis, single-stranded conformation polymorphism analysis, denaturing gradient gel electrophoresis (DGGE), or a combination thereof.

In another aspect, there is provided a method for producing a Cannabis plant with a powdery mildew resistant phenotype, comprising: identifying a first parent plant having a variant at a polymorphic site on chromosome 9 of the Cannabis genome, wherein the presence of the variant in one or both copies of the polymorphic site indicates that the plant has a powdery mildew resistant phenotype; crossing the first parent plant with a second parent plant to product F1 progeny; and identifying a first F1 progeny plant that has the variant at the polymorphic site and/or that has the powdery mildew resistant phenotype.

In some embodiments, at least one of the first parent plant and the first F1 progeny plant are identified as having the variant using any embodiment of the methods disclosed herein.

In some embodiments, the method further comprises: crossing the first F1 progeny plant to a second F1 progeny plant to produce F2 progeny; and identifying a first F2 progeny plant that has the variant and/or that has the powdery mildew resistant phenotype.

In some embodiments, the method further comprises: self-pollinating the first F1 progeny plant to produce F2 progeny; identifying a first F2 progeny plant that has the variant at the polymorphic site and/or that has the powdery mildew resistant phenotype.

In some embodiments, the method further comprises: backcrossing the first F1 progeny plant with the first parent plant or the second parent plant to produce F2 progeny; and identifying a first F2 progeny plant that has the variant and/or that has the powdery mildew resistant phenotype.

In another aspect, there is provided a method for producing a plant with a powdery mildew resistant phenotype, comprising: identifying a first parent plant having a variant at a polymorphic sit on chromosome 9 of the Cannabis genome, wherein the presence of the variant in one or both copies of the polymorphic site indicates that the plant has a powdery mildew resistant phenotype; crossing the first parent plant with a second parent plant to product F1 progeny; crossing a first F1 progeny plant to produce F2 progeny, wherein the first F1 progeny plant is crossed to one of: a second F1 progeny plant, the first parent plant, the second parent plant, and itself; and identifying a first F2 progeny plant that has the variant and/or that has the powdery mildew resistant phenotype.

In some embodiments, at least one of the first parent plant and the first F2 progeny plant are identified as having the variant using any embodiments of the methods disclosed herein.

In another aspect, there is provided a method for producing a Cannabis plant, comprising crossing a Cannabis plant produced according to any embodiments of the breeding methods disclosed herein to a second Cannabis plant having at least one other desired trait.

In some embodiments, the at least one other desired trait comprises a different powdery mildew resistance phenotype.

In another aspect, there is provided a Cannabis plant or a plant cell produced by any embodiment of the methods disclosed herein.

Other aspects and features of the present disclosure will become apparent, to those ordinarily skilled in the art, upon review of the following description of specific embodiments of the disclosure.

### BRIEF DESCRIPTION OF THE DRAWINGS

Some aspects of the disclosure will now be described in greater detail with reference to the accompanying drawings.

FIG. 1 is a flowchart of an example method for testing a Cannabis plant, according to some embodiments.

FIG. 2 is a flowchart of an example method for producing a Cannabis plant with a powdery mildew resistant phenotype, according to some embodiments.

FIG. 3 is a flowchart of another example method for producing a Cannabis plant with a powdery mildew resistant phenotype, according to some embodiments.

FIG. 4 is a histogram of clone-assay-derived disease index (DI) of a Cannabis germplasm population, where 0% DI indicates full resistance and 100% indicates full susceptibility. The dashed lines indicate the mean (M) and median (Mdn) and the solid lines indicate candidates of highly resistant (HR), moderately resistant (MR), and susceptible(S) phenotypic groups.

FIG. 5 is a photograph of a powdery mildew-resistant genotype N88 (bottom) compared to a susceptible(S) genotype (top) at 10 wpi.

FIG. 6 is a photograph of F1 progeny of a powdery mildew resistant genotype W03 (right) compared to a susceptible(S) genotype (left) at 4 wpi.

FIG. 7A is a microscopic image of a leaf of a susceptible(S) genotype plant at 2 wpi, showing conidiophores (circled) and a mycelial network of G. ambrosiae.

FIG. 7B is a microscopic image of a leaf of a powdery mildew resistant genotype W03 plant at 4 wpi stained with trypan blue, showing no visible conidiophores.

FIG. 8 is a photograph showing a leaf of a powdery mildew resistant genotype W03 plant, compared to a leaf of a susceptible genotype plant(S), and a leaf of a powdery mildew resistant plant with a marker showing close proximity to a previously reported R gene (PR). Panel A is a microscopic image of the leaf of the W03 plant and panel B is a microscopic image of the leaf of the PR plant.

FIG. 9 is a graph depicting the results of a genome wide association study (GWAS) analysis for powdery mildew resistance using log transformed DI as the phenotype.

FIG. 10 is graph depicting a detailed view of chromosome 3 from the GWAS analysis of FIG. 9. An SNP associated with PM resistance designated MR54 is circled (SNC_044372.1_82694448).

FIGS. 11A and 11B are allelic discrimination plots from KASP assays of clones (FIG. 11A) and adult plants (FIG. 11B) using the MR54 marker to test F1 progenies from crosses between the N88 genotype and a susceptible cultivar, showing discrimination between heterozygous and homozygous plants.

FIGS. 12A and 12B are box plots showing a comparison of clonal (FIG. 12A) and adult plant (FIG. 12B) DI values between a control group and PM resistant genotypes confirmed using the MR54 marker.

FIGS. 13A and 13B are scatter plots depicting bulk segregant analysis (BSA) results using Bayesian implementation to estimate SNP probabilities on a W03×AC F1 population and a N88×AC F1 population, respectively, both segregating for powdery mildew resistance. The analyses were conducted using the Pink Pepper reference genome.

FIGS. 14A and 14B are scatter plots depicting BSA results using Euclidean distance (ED) metric on a W03×AC F1 population and a N88×AC F1 population, respectively, both segregating for powdery mildew resistance. The analyses were conducted using the Pink Pepper reference genome. The ED values for each SNP have been raised to the 4^{th} power to increase signal to noise ratio.

FIGS. 15A and 15B are scatter plots depicting BSA results between base positions 50,000,000 and 62,000,000 of chromosome 9 of the Pink Pepper genome. FIG. 15A shows the Bayesian metric derived probabilities of SNPs linked to PM resistance and FIG. 15B shows the Euclidian distances of SNPs between bulks raised to the 4^{th} power. The top half shows the SNPs from the W03×AC F1 population and the bottom half shows the SNPs from the N88×AC F1 population.

FIGS. 16A and 16B are scatter plots depicting BSA results between base positions 50,000,000 and 62,000,000 of chromosome 9 of the CBDRx genome. FIG. 12A shows the Bayesian metric derived probabilities of SNPs linked to PM resistance and FIG. 12B shows the Euclidian distances of SNPs between bulks raised to the 4^{th} power. The top half shows the SNPs from the W03×AC F1 population and the bottom half shows the SNPs from the N88×AC F1 population.

FIGS. 17A to H are allelic discrimination plots of PACE genotyping assays in F1 populations: (A) MR110 in N88/AC; (B) MR110 in W03/AC (samples #1-88); (C) MR110 in W03/AC (samples 89-176); (D) MR121 in W03/AC; (E) MR124 in N88/AC; (F) MR125 in N88/AC; (G) MR131 in N88/AC; (H) MR54 in W03 F1. Heterozygous resistant and homozygous susceptible plants are as indicated.

FIGS. 18A-G are allelic discrimination plots of PACE genotyping assays in F2 populations: (A) MR110 in N88 F2; (B) MR110 in W03 F2; (C) MR121 in W03 F2; (D) MR124 in N88 F2; (E) MR125 in N88 F2; (F) MR131 in N88 F2; (G) MR54 in W03 F2 and N88 F2. Heterozygous resistant (R), homozygous resistant (R), and homozygous susceptible(S) are as indicated.

### DEFINITIONS

As used herein and in the appended claims, the singular forms of “a”, “an”, and “the” include plural referents unless the context clearly dictates otherwise.

“Allele” as used herein refers to one of two or more alternative nucleic acid sequences at a genetic locus, such as a polymorphic site. As Cannabis has a diploid genome, there are two copies of every genetic locus. A plant that is “homozygous” has the same allele at both copies of the locus, whereas a plant that is “heterozygous” has one allele at one copy of the locus and a different allele at the other copy.

“Cannabis” as used herein is inclusive of all species and varieties falling within the genus Cannabis, whether Cannabis sativa, Cannabis indica, or Cannabis ruderalis, and whether or not the variety is “drug” (i.e., contains appreciable levels of the psychoactive cannabinoid tetrahydrocannabinol, “THC”) or “non-drug” (i.e., contains less than about 0.2% or 0.3% by dry weight of THC, e.g., “hemp”). All Cannabis genomes have 10 chromosomes, including the sex chromosome. However, different Cannabis genome assemblies use different chromosome numbering systems. As such, equivalent or homologous chromosomes may be referred to with different numbers. For example, the Purple Kush chromosome 5 (identified by GenBank as CM010796.2) is equivalent or homologous to CBDRx chromosome 1 (identified by NCBI as NC_044371.1) with the only difference that the sequence is inverted. Herein, reference is made to the Pink Pepper genome (assembly ASM2916894v1, GenBank GCA_029168945.1) and the CBDRx genome (assembly cs10, GenBank GCA_900626175.2).

“Cultivar” and “variety” are used interchangeably herein to refer to a group of plants that is substantially distinct, stable, and uniform in its characteristics when propagated. It will be understood to a person skilled in the art that individual plants of a given variety or cultivar may be genetically similar but may not be genetically identical.

An “endogenous” gene as referred to herein refers to a gene that is naturally present in a population of Cannabis plants and has not been introduced through genetic modification.

“Genetic marker” as used herein refers to a polymorphic site that is associated with a phenotype or trait such that inheritance of particular allele of the genetic marker is indicative of the presence or absence of the phenotype/trait.

“Genotype” as used herein refers to the genetic constitution of an individual plant (or group of plants) at one or more genetic loci. When used in reference to a Cannabis plant, “genotype” may be used interchangeably with “variety” or “cultivar”.

“Identity” as used herein refers to sequence similarity between two polynucleotide molecules. Identity can be determined by comparing each position in the aligned sequences, for example, using BLAST (Basic Local Alignment Search Tool). A degree of identity between nucleic acid sequences is a function of the number of identical or matching nucleic acids at positions shared by the sequences over a specified region.

“Linkage” as used herein refers to the co-inheritance of alleles at two polymorphic sites due to the proximity of the polymorphic sites on the same chromosome.

“Linkage disequilibrium” as used herein refers to a situation where two alleles of respective polymorphic sites segregate in a non-random manner i.e., have a recombination frequency of less than 50%. The determination of the allele at one polymorphic site can provide the identity of the allele at another polymorphic site in linkage disequilibrium therewith.

“Nucleotide sequence”, “polynucleotide sequence”, “nucleic acid” or “nucleic acid molecule” as used herein refers to a DNA or RNA polymer which can be single or double stranded and optionally contains synthetic, non-natural, or altered nucleotide bases capable of incorporation into the DNA or RNA polymer. For a given nucleotide sequence, the acronyms A, C, G, and T are used to refer to adenine, cytosine, guanine, and thymine, respectively. The “complement” of a given DNA sequence is the sequence of the opposing strand based on nucleotide pairing rules (i.e., A pairs with T and C pairs with G). The sequences herein also use the IUPAC (International Union of Pure and Applied Chemistry) codes R, Y, S, W, K, and M to represent two possible nucleotides. These codes have their IUPAC-recognized meanings, whereby R indicates A or G, Y indicates C or T, S indicates G or C, W indicates A or T, K indicates G or T, and M indicates A or C.

“Plant material” as used herein refers to any portion or combination of portions of a Cannabis plant including, but not limited to, seeds, stems, stalks, leaves, flowers, roots, whether fresh or dry, or whether intact, cut, or comminuted.

“Polymorphism” or a “polymorphic site” as used herein refers to a position within a given genomic region at which variation exists within a population. A “polymorphism” or “polymorphic site” is the occurrence of two or more forms (alleles) at a position in the genome within a population, in such frequencies that the presence of the rarest of the forms cannot be explained by mutation alone. Polymorphic sites may occur in both coding regions and noncoding regions of genes or in intergenic regions. Polymorphic sites may involve a single nucleotide polymorphism (SNP) or may involve an insertion or deletion (“indel”), or rearrangement.

“Powdery mildew” (also abbreviated as “PM”) as used herein refers to a fungal disease characterized by development of a powdery appearance on the leaf surface of an infected plant. PM can be caused by multiple different species including, but not limited to, species of the Golovinomyces genus such as G. ambrosiae and G. cichoracearum.

“Powdery mildew resistance” and “a powdery mildew resistance phenotype” as used herein refer to a trait in which a plant having the trait is less affected by PM compared to a plant without the trait (i.e., a “susceptible” plant). For example, a plant with PM resistance may display reduced growth and/or reproduction of the fungal PM pathogen and may have less severe (or no) infection symptoms, compared to a PM susceptible plant infected at the same time and grown under the similar conditions. A plant displaying “complete” PM resistance is one showing no signs of infection following exposure to PM under conditions in which a susceptible plant would become infected. A plant displaying “partial” PM resistance is one showing reduced signs of infection compared to a susceptible plant.

“Primer” as used herein refers to an oligonucleotide capable of annealing to a nucleic acid template and providing a 3′ end that serves as a substrate for extension by a polymerase or reverse transcriptase. As used herein, “forward primer” and “reverse primer” are relative terms to refer to primers that anneal to opposite strands of the nucleic acid template.

“Polymerase chain reaction” or “PCR” as used herein refers to in vitro amplification of at least one target nucleic acid sequence by repeated cycles of denaturation and polymerase-mediated extension of primers.

“Quantitative Trait Locus” or “QTL” as used herein refer to a region of the genome that contains one or more genes or other sequences that are associated with a specific phenotype.

A “single nucleotide polymorphism” (“SNP”) as used herein refers to a polymorphism that involves a variation at a single nucleotide position.

A “variant” or “variant allele” as used herein refers to an allele at specific polymorphic site that varies from the most frequently occurring allele at that site and/or the allele at that site in a reference genome (the “reference allele”).

### DETAILED DESCRIPTION

Generally, the present disclosure provides genetic markers for powdery mildew resistance in Cannabis and related methods. The genetic markers can be used for testing and selective breeding of Cannabis plants. The genetic markers may be used to select for Cannabis plants with a powdery mildew resistance phenotype.

In some embodiments, the genetic marker may be on chromosome 9 of the Cannabis genome. With reference to the Pink Pepper genome, in some embodiments, the genetic marker may be located within a region on chromosome 9 (NC_083609.1) between about 57 megabases to about 60 megabases, between about 57.4 to about 59.5 megabases, between about 58 to about 59 megabases, between about 58.1 to about 58.6 megabases, or between about 58, 164, 118 and about 58,552,299 bases. With reference to the CBDRx genome, in some embodiments, the genetic marker may be located within a region on chromosome 9 (NC_044376.1) between about 56 megabases to about 60 megabases, between about 56.2 to about 59.2 megabases, between about 58 to about 59 megabases, between about 58.1 to about 58.6 megabases, or between about 58, 108,801 to about 58,570,898 bases. It will be understood that reference to specific base positions of the Pink Pepper and CBDRx genomes are intended to be inclusive of equivalent base positions of other Cannabis genomes.

In some embodiments, the genetic marker is a SNP. In some embodiments, the SNP is one of the SNPs in Table 1.

TABLE 1

| Marker | Chromosome_Position | Chromosome_Position |
|---|---|---|
| (SNP) | (Pink Pepper) | (CBDRx) |
| MR110 | NC_083609.1_58448827 | NC_044376.1_58108802 |
| MR121 | NC_083609.1_58552298 | NC_044376.1_58570897 |
| MR124 | NC_083609.1_58289703 | NC_044376.1_58423201 |
| MR125 | NC_083609.1_58164119 | NC_044376.1_58314046 |
| MR131 | NC_083609.1_58289715 | NC_044376.1_58423213 |

Table 2 provides the genomic context sequences around each of the SNPs from Table 1. The underlined nucleotide in each sequence is the reference allele and the variant is as indicated.

TABLE 2

| Marker | SEQ | Position | | |
|---|---|---|---|---|
| (SNP) | ID NO. | in SEQ | Genomic Context Sequence | Variant |
| MR110 | 1 | 101 | CGATGATCAAGATACTTGCTAAGGAGCTRAAGGGGAYGGGGA | A |
| | | | TCACGGCCAACTGCKTGGCMCCGGGACCAATCGCGACSGATA | |
| | | | TGTTCTTTTCGGGGAAGAGTGAGGATGACTTGAAGAAAGTGG | |
| | | | TGGATGAGTGCCCCATGGGGAGGCTTGGAGAGGTTAAGGATG | |
| | | | TGGTTCCWGTGGTGGGATTCTTGGCTGGTGAT | |
| MR121 | 2 | 101 | CATGCAAGTAGTGGTGTAAATGGCTTTCAACTGGGAATGATT | A |
| | | | CATATAAGAACTCCTTGTAATATTCTTTACGAGGTGCATGGC | |
| | | | AGAGGATCACACATTTCCCGGAATTATCTAGCAATGGCCGAC | |
| | | | TAGCATGACCCATCATCTGCAACAGATCCGCGACAGGATAAT | |
| | | | CTGTGTGGACATTTTCACGGCCATCATAGTAT | |
| MR124 | 3 | 101 | TTTGATYGATTMTTTTTWCAAACCTGTAATTAATTCTTCGAG | G |
| | | | AACAAGTAACYAAAAYCTTCTCTTTGTTCCTGAAAAACGGAG | |
| | | | TTGATTCAGTTTCTGSTTCTGCTTCATTYTTCTTCTCTTTCC | |
| | | | AACMCAAAAGWGTCCTTTKAGGCCTCTCTGCAGCTGTATCAT | |
| | | | CCTTCTTCACTGGTGCCACCACCACAGTCTCA | |
| MR125 | 4 | 101 | AAWTGCATTGTTGATTATTTTCAAATTTTCTAATTCTAAATA | T |
| | | | GGTTTGTGCATGGAGATGTAAAGCCCGAGAATTTTTTACTTG | |
| | | | GTCAACCTGGAACAGTCGATGAGAAGAAACTATATCTTGTTG | |
| | | | ATCTTGGAYTGGGTATTGTTTCTTTTCCGASTTCTTATCCTT | |
| | | | TYAACCGTGTCATTTTTYTGAATTTTCTCACA | |
| MR131 | 5 | 101 | TTTTTWCAAACCTGTAATTAATTCTTCGAGAACAAGTAACYA | T |
| | | | AAAYCTTCTCTTTGTTCCTGAAAAACGGAGTTGATTCAGTTT | |
| | | | CTGSKTCTGCTTCATTCTTCTTCTCTTTCCAACMCAAAAGWG | |
| | | | TCCTTTKAGGCCTCTCTGCAGCTGTATCATCCTTCTTCACTG | |
| | | | GTGCCACCACCACAGTCTCACTGTGCTTTCTC | |

The sequences in Table 2 are shown in the 5′ to 3′ direction. It will be understood that the variants in the complement sequences will follow base pairing rules such that the variant in the complement for SNPs MR110, MR121, MR124, MR125, and MR13 will be T, T, C, A, and A, respectively.

It will also be understood that the genomic context sequence flanking each SNP may vary slightly between different Cannabis varieties and cultivars. Each SNP may therefore be at an equivalent position in a sequence having at least 90% identity, at least 95%, at least 97%, or at least 99% identity to the SEQ ID NOS. 1-5 in Table 2 and reference to a given SNP is intended to be inclusive of such equivalent positions.

In other embodiments, the genetic marker may be one of the polymorphic sites in Table 3 below. The positions of each polymorphic site are with reference to chromosome 9 of the Pink Pepper genome (NC_083609.1). For SNPs, the Reference>Variant nucleotides are shown. For insertions, deletions, and duplications, the inserted (ins), deleted (del), or duplicated (dup) sequence is shown.

TABLE 3

| | Type of | |
|---|---|---|
| Position | Polymorphism | Variant |
| 57439234 | SNP | G > C |
| 57448607 | SNP | G > A |
| 57448824 | SNP | G > T |
| 57449063 | SNP | G > A |
| 57572664 | Deletion | delGTAAGT |
| 57573450 | SNP | G > C |
| 57727314 | SNP | G > C |
| 57727339 | SNP | A > G |
| 57727450 | SNP | G > A |
| 57727666 | Deletion | delCAT |
| 57727702 | SNP | A > G |
| 57830630 | Insertion | insCCACCAATGGTTGCAACTCCACA [SEQ ID NO. 13] |
| 57868026 | SNP | C > T |
| 57868110 | SNP | C > A |
| 57868138 | SNP | C > T |
| 57893405 | Deletion | delAAAG |
| 58151984 | SNP | A > T |
| 58151993 | SNP | G > A |
| 58163609 | Deletion | delTCTGGTGAGATTTGATATTGATAC [SEQ ID NO. 14] |
| 58220890 | SNP | T > C |
| 58220985 | SNP | C > T |
| 58221001 | SNP | C > T |
| 58232858 | SNP | A > C |
| 58232859 | SNP | C > A |
| 58232914 | Insertion | insTCT |
| 58232939 | SNP | C > A |
| 58233215 | SNP | C > G |
| 58271194 | SNP | G > A |
| 58271197 | SNP | A > G |
| 58271205 | Insertion | insGC |
| 58271207 | Deletion | delAA |
| 58271245 | SNP | A > G |
| 58271249 | SNP | C > T |
| 58271262 | Duplication | dupTTA |
| 58271283 | SNP | C > G |
| 58271401 | SNP | A > G |
| 58271554 | SNP | G > C |
| 58279575 | SNP | G > C |
| 58279592 | SNP | T > G |
| 58279625 | SNP | T > G |
| 58279632 | SNP | T > C |
| 58279634 | SNP | G > C |
| 58351094 | Duplication | dupCCACCA |
| 58354072 | SNP | A > C |
| 58354117 | SNP | C > G |
| 58354122 | SNP | C > T |
| 58354690 | SNP | C > T |
| 58403005 | Insertion | insA |
| 58404720 | SNP | C > A |
| 58405905 | SNP | G > A |
| 58406536 | SNP | A > T |
| 58448093 | SNP | G > T |
| 58448300 | SNP | C > A |
| 58450356 | SNP | A > C |
| 58451619 | SNP | C > T |
| 58451678 | SNP | G > T |
| 58451741 | SNP | G > A |
| 58551005 | SNP | C > G |
| 58555952 | SNP | G > A |
| 58556597 | SNP | G > A |
| 58611682 | Deletion | delGTTGGTGCTTTACTTACTGGGTTGGTTTTTGTTTTTGTC |
| | | TTTCAAGTGTTTGCTTATATGTCTTGTTTTTTTCTTTTTATG |
| | | CAG [SEQ ID NO. 15] |
| 58656390 | Insertion | insAGTTTTTTCCCCATCTGGTGTTTCTAGCATGCAATGGAA |
| | | GACAGGAGCTGAACCA [SEQ ID NO. 16] |
| 58657944 | SNP | C > A |
| 58683067 | SNP | C > T |
| 58683256 | SNP | G > A |
| 58683761 | SNP | C > A |
| 58723280 | SNP | G > C |
| 58876193 | Duplication | dupG |
| 58878897 | SNP | T > A |
| 59177284 | Deletion | delACAAGTAAGTTGTTCAACAAGCTCAATATCTTTGCTTTT |
| | | GCTTTGTTTAAATCTTCGAAAGATGAATTCATAAGT |
| | | [SEQ ID NO. 17] |
| 59224781 | SNP | C > T |
| 59224873 | SNP | C > G |
| 59280039 | Deletion | delGTACTCTATTTATTCATGTATAATAATAAAAACTCCCCT |
| | | TTTTTGTTAATTCCACCAATTTGAACTTTTAAACGGCTTTT |
| | | CTCTGCAG [SEQ ID NO. 18] |
| 59347779 | SNP | T > A |

Other genetics markers of PM2 are also possible, including other polymorphic sites in linkage disequilibrium with at least one of MR110, MR121, MR124, MR125, and MR131 or in linkage disequilibrium with one of the polymorphic sites in Table 3. In some embodiments, the other polymorphic site may be another SNP. In other embodiments, the other polymorphic site may be any other suitable polymorphism.

MR110, MR121, MR124, MR125, and MR131 may each be used as a genetic marker for powdery mildew resistance in Cannabis plants, alone or in combination. Without being limited by theory, as discussed in the Examples below, these markers are believed to be in linkage disequilibrium with a single locus with a dominant Mendelian mode of inheritance that the inventors have designated “PM2”.

The presence of a variant at a given SNP may indicate that a Cannabis plant has a powdery mildew resistant phenotype. The PM resistant Cannabis plant may display complete or partial resistance. As the inheritance of PM2 appears to be dominant, the presence of the variant at one or both copies of the SNP site may be indicative of the powdery mildew resistant phenotype. In other words, a powdery mildew resistant Cannabis plant can be either heterozygous or homozygous for the variant. Conversely, the absence of a variant at a given SNP may indicate that a Cannabis plant is susceptible to powdery mildew or that the plant has a different PM resistance mechanism.

As demonstrated in the Examples below, observed PM2-mediated resistance appears to be strong and durable, remaining effective during the full growth cycle of Cannabis. Microscopic analysis revealed limited mycelial growth and complete suppression of the sporulation stage of the fungal PM pathogen even after several weeks post-infection. The PM2 locus appears to confer resistance to multiple different isolates of powdery mildew that were collected at different production facilities. Therefore, the genetic markers disclosed herein can be used for marker-assisted selection (MAS) based breeding methods, or other breeding means, to introduce the PM2 locus into Cannabis cultivars to provide strong resistance to powdery mildew. In some embodiments, PM2 may be introduced into PM-susceptible cultivars that lack any other R genes. In other embodiments, PM2 may be introduced into cultivars that already have one or more other R genes (known as a “stacking” approach), which may provide advantages such as stronger protection against a broader range of powdery mildew strains and/or increased durability of the resistance.

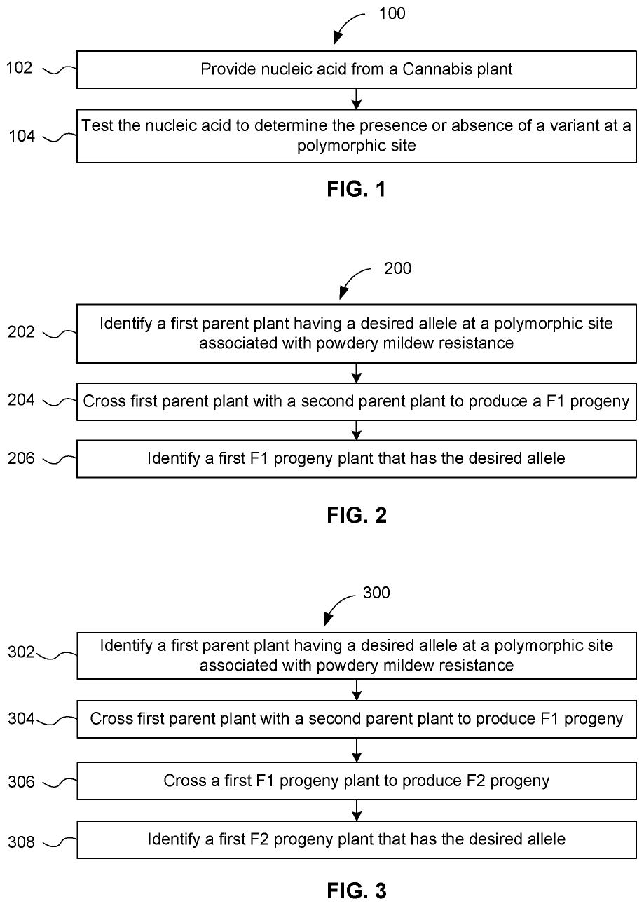

FIG. 1 is a flowchart of an example method 100 for testing Cannabis plants, according to some embodiments.

At block 102, nucleic acid from a Cannabis plant may be provided. The term “provide” in this context refers to extracting, obtaining, or otherwise acquiring the nucleic acid. In some embodiments, the nucleic acid may be genomic DNA. In other embodiments, the nucleic acid may be RNA, cDNA, or any other suitable nucleic acid that contains the genetic marker of interest.

In some embodiments, Cannabis plant material may be provided and the nucleic acid may be extracted therefrom. The plant material may comprise a whole Cannabis plant or may comprise a portion thereof, such as at least a portion of the floral tissue, leaves, stems, roots, or a combination thereof. In other embodiments, the Cannabis plant material may comprise one or more seeds. The nucleic acid may be extracted using any suitable extraction technique including, but not limited to, organic extraction, silica column-based extraction, magnetic separation, anion exchange technology, salting out, and/or cesium chloride density gradients. In other embodiments, the nucleic acid may be provided in a purified form, having been previously extracted from Cannabis plant material.

At block 104, the nucleic acid may be tested to determine the presence or absence of a variant at a polymorphic site. In some embodiments, the polymorphic site may be a polymorphic site on chromosome 9 (using the numbering of the Pink Pepper and CBDRx genomes), including a site on chromosome 9 within any of the base pair ranges discussed above. In some embodiments, the polymorphic site may be a SNP. In some embodiments, the SNP may be one of the SNPs in Table 1 and the variant may be the respective variant for that SNP as listed in Table 2. In other embodiments, the polymorphic site may be another polymorphic site (e.g., another SNP) in linkage disequilibrium with one of the SNPs in Table 1. In other embodiments, the polymorphic site may be one of the sites in Table 3 or another polymorphic site in linkage disequilibrium therewith. The variant may be in one or both copies of the endogenous genomic DNA sequence. In other words, the Cannabis plant may be heterozygous or homozygous for the variant.

In some embodiments, testing the nucleic acid comprises nucleic acid amplification, e.g., amplification carried out by polymerase chain reaction (PCR). Nucleic acid amplification alone may detect the presence or absence of a given variant (e.g., using allele-specific primers) or may be done as a preliminary step for another analytical method.

The testing may performed using any suitable technique in the art for detecting a variant at a polymorphic site including, but not limited to, allele-specific amplification, allele-specific probe hybridization, allele-specific primer extension, sequencing, 5′ nuclease digestion, molecular beacon assay, oligonucleotide ligation assay, size analysis, single-stranded conformation polymorphism analysis, denaturing gradient gel electrophoresis (DGGE), or a combination thereof. In some embodiments, the testing may be done using competitive allele-specific PCR which utilizes two competitive allele specific forward primers and a common reverse primer. Examples of commercially available competitive allele-specific PCR assay include KASP™ and PACE™ genotyping assays. The primers for these assays may be designed based on one of SEQ ID Nos. 1-5 in Table 2 above for the relevant SNP for that sequence.

In some embodiments, the nucleic acid may be tested to determine the presence or absence of respective variants at two or more polymorphic sites, such as the variants of two or more of the SNPs in Table 1. The two or more polymorphic sites may be tested simultaneously or sequentially. In some embodiments, the two or more polymorphic sites may be tested in the same assay, for example, using multiplex PCR. In other embodiments, the two or more polymorphic sites may be tested in separate assays.

Thus, the method 100 may be used to determine the genotype of a Cannabis plant for a given genetic marker. The testing may also determine if the plant is heterozygous or homozygous for a specific variant. Such testing may be used to predict whether a Cannabis plant is resistant or susceptible to powdery mildew. The testing may be performed at any stage of development, from seed to adult plant. The method 100 may also be used to identify plants having a particular variant to aid in selective breeding methods, such as the methods 200 and 300 of FIGS. 2 and 3 discussed below.

FIG. 2 is a flowchart of an example method 200 for producing a Cannabis plant with a powdery mildew resistance phenotype.

At block 202, a first parent plant having a variant at a polymorphic site associated with powdery mildew resistance may be identified. In some embodiments, the polymorphic site may be a polymorphic site on chromosome 9 (using the numbering of the Pink Pepper and CBDRx genomes), including a site on chromosome 9 within any of the base pair ranges discussed above. In some embodiments, the polymorphic site may be a SNP. In some embodiments, the SNP may be one of the SNPs in Table 1 and the variant may be the respective variant for that SNP as listed in Table 2. In other embodiments, the polymorphic site may be another polymorphic site (e.g., another SNP) in linkage disequilibrium with one of the SNPs in Table 1. In other embodiments, the polymorphic site may be one of the sites in Table 3 or another polymorphic site in linkage disequilibrium therewith.

In some embodiments, the first parent plant may be identified as having the variant by performing the steps of the method 100 as discussed above. In other embodiments, the first parent plant may be identified by any other suitable method. In yet other embodiments, the genotype of the first parent plant at the polymorphic site may have been previously determined and the step of identifying the first parent plant may comprise selecting a plant with the variant.

At block 204, the first parent plant may be crossed with a second parent plant to produce F1 progeny. The second parent plant may be a plant of a different cultivar or variety than the first parent plant. In some embodiments, the second parent plant may be a plant that is powdery mildew susceptible. In other embodiments, the second parent plant may be a plant with a different PM resistance phenotype (e.g. associated with a different R gene). In some embodiments, the second parent plant may have one or more other desired traits (e.g., autoflowering, desired height, early or late maturity, resistance to a different disease, cannabinoid and/or terpene content, aroma, etc.). In other embodiments, the second parent plant may be any other suitable plant.

In some embodiments, the second parent plant may be identified as not having the variant at the given polymorphic site. For example, the second parent plant may be identified as having the reference allele at that polymorphic site. In other embodiments, the genotype of the second parent plant at the polymorphic site may not be known.

The first parent plant may be female and the second parent plant may be male (or vice versa) and the first and second parent plants may be crossed by pollinating the female plant with pollen from the male plant using any known cross-pollination technique. The F1 seed may then be collected. In some embodiments, the F1 seed may be used directly in block 206 below. In other embodiments, the F1 seed may be germinated and grown to produce one or more seedlings or adult plants at any suitable stage of maturity, and such seedlings or adult plants may then be used in the steps at block 206.

At block 206, a first F1 progeny plant may be identified that has the variant at the polymorphic site. The first F1 progeny plant may be identified as having the variant by performing the steps of the method 100 or by any other suitable method. In some embodiments, a plurality of F1 progeny plants may be screened to identify one or more plants that have the variant. The proportion of F1 progeny plants having the variant will be based on whether the first parent plant is homozygous or heterozygous for the variant. In embodiments in which the first parent plant is homozygous, all of the F1 progeny plants may be expected to have the variant. In embodiments in which the first parent plant is heterozygous for the variant, 50% of the F1 progeny plants may be expected to have the variant. In either case, the F1 progeny plants with the variant would all be expected to be heterozygous (assuming the second parent plant does not have the variant).

Alternatively, or additionally, the first F1 progeny plant may be identified as having the powdery mildew resistance phenotype. In these embodiments, the F1 progeny plant(s) may be grown to a suitable stage and challenged with powdery mildew, for example, using techniques outlined in the Examples below. In some embodiments, the first F1 progeny plant may be identified based on having a powdery mildew resistance phenotype, followed by testing to confirm that the F1 progeny plant has the variant (or vice versa).

The method 200 may further comprise using the first F1 progeny plant to produce one or more F2 progeny plants and identifying at least one F2 progeny plant that has the variant at the polymorphic site and/or that has the powdery mildew resistance phenotype. The F2 progeny plants may be tested for the presence of the variant using the method 100 or any other suitable method.

Various breeding techniques may be used to produce the F2 progeny. In some embodiments, the F2 progeny may be produced by identifying a second F1 progeny plant that has the variant and crossing the first F1 progeny plant with the second F1 progeny plant (“sib-crossing”).

In other embodiments, the F2 progeny may be produced by self-pollinating (“selfing”) the first F1 progeny plant. Cannabis plants are typically dioecious, with separate male and female plants. However, selfing can be accomplished by artificially reversing the sex of flowers from female to male (or vice versa) on some branches. For example, silver thiosulfate treatment may be used to masculinize some branches of a female plant to provide male flowers with pollen that can be used to pollinate female flowers of the same plant. In other embodiments, any other suitable technique may be used to reverse the sex of flowers on one or more branches of a Cannabis plant. In other embodiments, the F2 progeny may be selfed using any other suitable method.

In yet other embodiments, the F2 progeny may be produced by backcrossing the first F1 progeny plant to the first parent plant or the second parent plant. Backcrossing may utilize the same first or second parent plant as was used to produce the F1 progeny or a genetic clone or another plant of the same cultivar. In some embodiments, several generations of backcrossing to the second parent may be done in order to produce Cannabis plants that are genetically similar or substantially genetically identical to the second parent plant but with the addition of the variant (and PM2 locus) from the first parent plant. In this manner, the powdery mildew resistance phenotype from one Cannabis cultivar can be introduced into another Cannabis cultivar.

In some embodiments, a combination of the breeding techniques discussed above may be used. As one example, the first F1 progeny plant may be backcrossed to the first or second parent plant to produce BC1 (backcross 1) progeny, followed by selfing of one of the BC1 plants or sib-crossing of two BC1 plants with one another.

Although the breeding steps in method 300 above refer to a “first” F1 progeny plant, it will be understood that this is for simplicity only and any number of plants may be crossed to produce additional progeny.

The F2 progeny may include one or more plants that are heterozygous for the variant and, depending on the parent plants used in the cross, may also include one or more plants that are homozygous for the variant. In some embodiments, one or more of the heterozygous and/or homozygous F2 progeny plants may be selected for further breeding and/or for clonal propagation.

FIG. 3 is a flowchart of an alternative method 300 for producing a Cannabis plant with powdery mildew resistance.

At block 302, a first parent plant having a variant at a polymorphic site associated with powdery mildew resistance may be identified. At block 302, the first parent plant may be crossed with a second parent plant to produce F1 progeny. The steps at blocks 302 and 304 may be similar to the steps of blocks 202 and 204 of the method 200 as described above.

At block 306, a first F1 progeny plant may be crossed to produce F2 progeny. Unlike the method 200, the first F1 progeny plant may not be identified as having the variant at the polymorphic site prior to breeding. To produce the F2 progeny, the first F1 progeny plant may be crossed with one of: a second F1 progeny plant (i.e., sib-crossing), the first parent plant or the second parent plant (i.e., backcrossing), or itself (i.e., selfing) to produce F2 progeny.

At block 308, a first F2 progeny plant is identified that has the variant. The steps at block 308 may be similar to the steps at block 206 of the method 200 as described above.

The method 300 may be used, for example, in circumstances where the first parent plant is homozygous for the variant and thus all F1 progeny are assumed to be heterozygous for the variant. Heterozygous and/or homozygous F2 progeny can be used for further breeding and/or clonal propagation.

Also provided herein is a method comprising crossing a Cannabis plant produced by any embodiment of the methods 200 or 300 described above with a second Cannabis plant having at least one other desired trait. The Cannabis plant may be an F1 plant, an F2 plant, or any subsequent generation of plants produced by backcrossing, selfing, sib-crossing or any other suitable breeding technique. The other desired trait may be any other desired trait including, but not limited to, autoflowering, desired height, early or late maturity, resistance to a different disease, cannabinoid and/or terpene content, aroma, etc. In some embodiments, the other desired trait may be a different PM resistance phenotype (e.g. associated with a different R gene), thereby “stacking” PM resistance loci. The F1 progeny from the crossing of the Cannabis plant and the second Cannabis plant may then be selfed, back-crossed, sib-crossed, or bred in any other suitable manner to produce F2 progeny and subsequent generations of plants having both powdery mildew resistance and the other desired trait.

Also provided herein is a Cannabis plant or a plant cell produced by any embodiment of the selective breeding methods described above, including F1 plants, F2 plants, and any subsequent generation of plants produced by backcrossing, selfing, sib-crossing or any other suitable breeding technique. In some embodiments, the Cannabis plant may be in the form of a seed and the plant cell may be a seed cell. In other embodiments, the Cannabis plant may be a plant at any other stage of development and the plant cell may be any cell in any plant tissue at any development stage.

Also provided herein is plant material obtained from the Cannabis plant or plant cell. In some embodiments, the plant material comprises dried flower. In other embodiments, the plant material may comprise seeds produced by the Cannabis plant or plant cell.

Also provided herein is a Cannabis-derived product generated from the plant material. The term “Cannabis-derived product” in this context may refer to any substance comprising, or produced from, Cannabis plant material. In some embodiments, the Cannabis-derived product may comprise one or more of an extract, a resin, a concentrate, an isolate, and an oil. In some embodiments, the Cannabis-derived product may further comprise a carrier oil. In some embodiments, the carrier oil comprises a medium-chain triglyceride (MCT) oil (e.g., coconut oil, vegetable oil, olive oil, etc.).

Also provided herein is a composition comprising the Cannabis-derived product. In some embodiments, the composition may be in the form of a pharmaceutical product, a veterinary product, a natural health product, a dietary supplement, a food product, a beverage product, a cosmetic product, a topical product, a tincture, and/or a vaporizable product for use with a vaporizer.

In some embodiments, the composition may further comprise at least one pharmaceutically and/or nutritionally acceptable excipient. Non-limiting examples of suitable excipients include fillers, binders, carriers, diluents, stabilizers, lubricants, glidants, coloring agents, flavoring agents, coatings, disintegrants, preservatives, sorbents, sweeteners and any other pharmaceutically or nutritionally acceptable excipient. In other embodiments, the composition may further comprise any other ingredient or combination of ingredients.

Without any limitation to the foregoing, the genetic markers and methods are further described by way of the following examples. However, it is to be understood that these examples are for illustrative purposes only and should not be used to limit the scope of the present disclosure in any manner.

### Examples

### Example 1—Germplasm Screening for PM Resistance

A Cannabis germplasm collection containing 505 genotypes including production cultivars, landraces and exotic lines, was used for germplasm screening. This population was referred to as “CanD” for Cannabis Diversity. All 505 genotypes of the CanD germplasm collection were initially tested for resistance to powdery mildew using a clone-based infection assay. Clone assays were performed on all CanD genotypes with at least 6 replicates per genotype which were clonally propagated and rooted for 14 days in rockwool cubes. Inoculations were done by heavily “dusting” the plantlets at 3 weeks after cloning with fresh PM spores harvested from highly infected adult plants. Disease evaluation and scoring was performed at 4 weeks post inoculation (wpi). Plants were grown at 23° C. with relative humidity kept around 80% for the first 48 h (to ensure high level of spore germination), and then kept at 70% during the rest of the infection trial. Photoperiod was 16 h of light and 8 h of dark during vegetative growth for the duration of the clone assay.

Disease symptoms were evaluated in the three most infected leaves of three most infected replicates of each genotype at 4 wpi. Disease severity was assessed as “disease index” following a method previously developed (Seifi et al., 2013; Seifi et al., 2021) using a qualitative scale from zero to four where: 0 indicates healthy leaves with no signs of infection; 1 indicates leaves with few sporulated colonies of the pathogen covering less than 25% of the leaf; 2 indicates leaves where the fungus sporulation covered between 25-50% of the surface area of the leaf; 3 indicates leaves where between 50-75% of the leaf surface was covered with the fungus; and 4 indicates leaves where between 75-100% of the leaf surface was covered with the fungus.

The disease severity index was estimated for each replicate using the following formula (I):

$$DI = \frac{( 0 \times {n}_{0} ) + ( 1 \times {n}_{1} ) + ( 2 \times {n}_{2} ) + ( 3 \times {n}_{3} ) + ( 4 \times {n}_{4} )}{N \times 4} \times 100 \qquad ( I )$$

where DI indicates the disease severity in percentage; n₀, n₁, n₂, n₃, n₄ are the number of leaves with disease scores of 0, 1, 2, 3 and 4 respectively; and N is the total number of leaves evaluated.

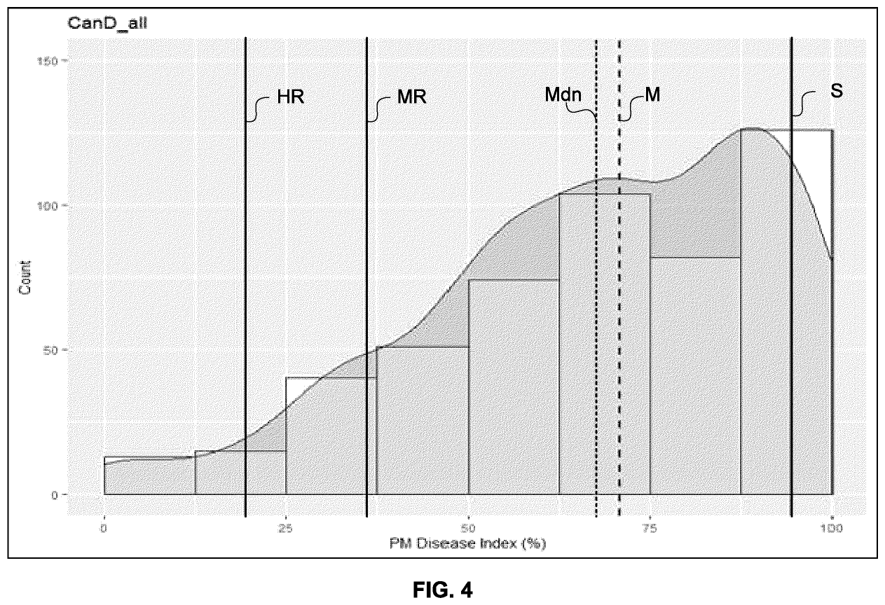

FIG. 4 shows the (left-skewed) distribution histogram of PM disease severity rates (disease index, DI) of the entire CanD population subjected to the clone assay. The continuous nature of the distribution of DI in the CanD population suggests that multiple loci can contribute to PM resistance in Cannabis.

The results of the clone-assay screening were then validated by performing an adult plant assay on a panel of 90 candidate genotypes selected from three different phenotypic groups of highly resistant (HR: DI≤33, comprising of ~5% of the population which were all included), moderately resistant (MR: 33<DI<66) and susceptible (S: 66<DI≤100). The adult plant assays were performed in a similar manner to the clone assays described above but on fully grown plants following a complete 12-week production cycle until the end of the flowering stage. The growth conditions were similar with the photoperiod being 16 h light/8 h dark for the first week and then changed to 12 h light/12 h dark for 8 weeks. The disease pressure during the adult plant experiments was significantly higher than that of clone assay, due to the longer time (12 weeks vs. 4 weeks) that plants were grown under constant disease pressure, as well as the high level of inoculum and re-infection with fresh spore which occurred during the infection trial. In severe susceptibility cases, sugar leaves of the flowers, petioles and parts of the stem tissue were also colonized by the PM pathogen. Disease severity at 12 wpi was evaluated using the DI index as described above.

A final group of 14 resistant genotypes (DI<50) were selected after the adult-plant infection trial. Out of 14 genotypes, 8 genotypes showed strong resistance responses to PM (DI<33), despite the high disease pressure. Furthermore, different isolates of PM, collected from different production facilities, were tested on the selected panel of highly resistant genotypes to assess the possibility of putative PM pathotypes that may be capable of breaking the observed resistance. However, none of the collected PM isolates were able to cause infection on any of the resistant genotypes in the panel, indicating that a single pathotype was present.

### Example 2—Mode of Inheritance of the Observed R Locus

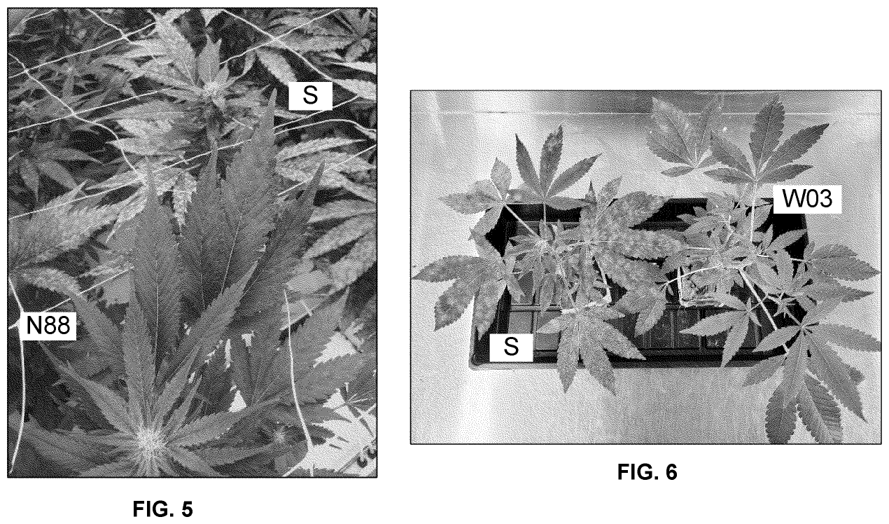

Several infection trials using the clone assay were performed on 12 different F1 populations derived from test crosses between highly resistant (HR) and susceptible(S) genotypes with the goal of determining the mode of inheritance of PM resistance. Among the 12 populations tested, 2 different HR genotypes, labeled as W03 and N88, consistently exhibited a 1:1 ratio for HR: S in their F1 progenies from the cross with a susceptible cultivar named “Alien Cake” (AC), indicating that resistance to PM in these crosses is mediated by a single dominant locus, and that the resistant parents are heterozygous for that trait. Hereafter, this dominant locus is referred to as “PM2”. FIGS. 5 and 6 are photographs of PM-resistant F1 progeny plants of the N88×AC and W03×AC crosses, respectively, showing no visible PM spots at 10 wpi (N88) and 4 wpi (W03) compared to susceptible plants.

Table 4 below shows the Chi-squared tests for goodness of fit for the N88×AC and W03×AC cross populations.

TABLE 4

| | HR-progeny | | S-progeny | | Chi-square test | | |
|---|---|---|---|---|---|---|---|
| Cross (HR × S) | Exp. Vs. Obs. | | Exp. Vs. Obs. | | χ² | df | P-value |
| N88 × AC | 140 | 136 | 140 | 138 | 0.007 | 1 | 0.93 |
| W03 × AC | 24 | 24 | 24 | 21 | 0.10 | 1 | 0.90 |

A heterozygous single dominant locus is expected to generate a 1:1 segregation in its progeny. As shown in Table 3, the p-values indicate that deviations from the expected 1:1 ratio are not significant (α=0.05). The progeny in the remaining F1 populations tested did not show strong PM resistance, suggesting a multigenic pathogen resistance in the parents of those crosses.

### Example 3—Microscopic Analysis of PM2-Mediated Resistance Response

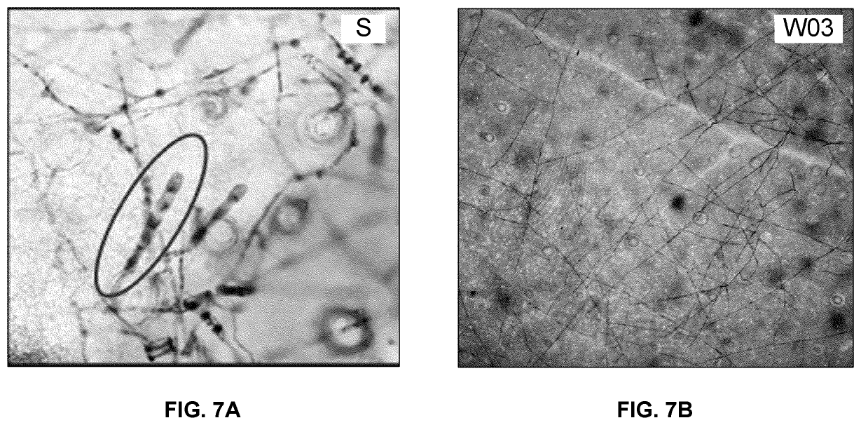

In susceptible Cannabis genotypes, the PM fungal pathogen G. ambrosiae can fully develop its mycelial network and conidiophores within 2 wpi, completing its infectious life cycle (see FIG. 7A). However, genotypes with PM2-mediated resistance show restricted mycelial growth and strong suppression of the conidiophore formation (see FIG. 7B), which is a key developmental stage for the sporulation phase of the pathogen. As shown in FIG. 7B, no conidiophores are present on an inoculated W03 leaf at 4 wpi.

### Example 4—Comparison of PM2-Mediated Resistance Response to Alternative PM Resistance Mechanism

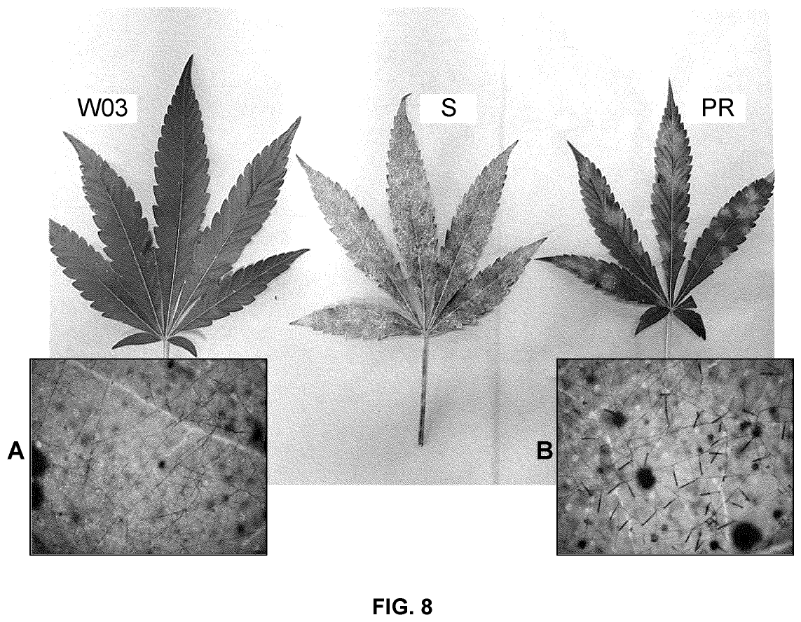

The resistance response in a PM2-harboring genotype was compared to a cultivar containing genetic markers in close proximity to a previously reported R gene (hereafter referred to as “PR”). As shown in FIG. 8, the PM2 genotype and the PR genotype showed clear differences in infection symptoms as well as growth and reproduction of the fungal pathogen. In the PM2 plant, some mycelial network development was observed, but without any detectable conidiophores, indicating that conidiophore formation and sporulation are suppressed (see panel A in FIG. 8). In contrast, in the PR plant, conidiophores were observed in sporadic patches of restricted PM colonies (see panel B in FIG. 8). Thus, the two PM resistance loci appear to induce two distinct defense mechanisms, with PM2 conferring stronger resistance.

### Example 5—Genome Wide Association Study (GWAS)

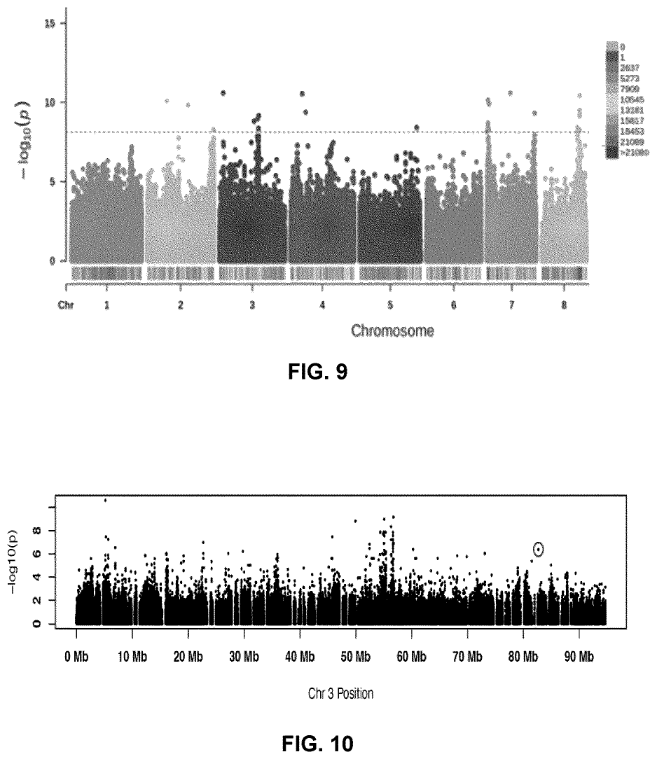

A genome wide association study (GWAS) for PM resistance was performed, using normalized DI and un-filtered SNPs (no minimum frequency filtering). Association mapping was performed with a model of the rMVP R Library (https://www.sciencedirect.com/science/article/pii/S1672022921000504) using 477 individuals and 6,991,319 SNP markers. To control for population structure, 5 principal components of the genotype data were included in the mixed-linear model (MLM). A log transformation was applied to DI phenotype data prior to input. The SNP data set was generated with whole genome sequencing data from the 477 CanD samples based on the CS10 reference genome. SNP calling was performed following best practices of Genome Analysis Tool Kit v 4.1.4.1 (GATK). The analysis identified several regions in the Cannabis genome significantly associated with DI (FIG. 9).

### Example 6—MR54 Marker Development and Validation

PACE assays were used to genotype the segregating F1 populations of the N88 and W03×AC crosses and determine associations between SNPs and the PM resistance phenotype. None of the significant SNPs from the GWAS analysis showed association with the PM resistance phenotype in the F1 populations with a 1:1 ratio of resistance/susceptible phenotypes. The lack of associations between significant SNPs and the resistance phenotype was expected since the power of GWAS analysis to detect associations is greatly affected by the frequency of causal alleles and only two genotypes out of 505 showed heritable, single-loci and dominant PM resistance.

The following rationale can be used to try to map an allele with low frequency in a population: (1) PM resistance can be associated with a SNP that is heterozygous in the parents of the two F1 populations with 1:1 susceptible/resistant ratios; (2) GWAS analysis of DI probably could be weaker association but above baseline level (i.e. compared to non-associated SNPs); and (3) the SNPs survey all of the regions in the genome with these weak associations (at least 1 SNP in each chromosome segment with an association). Applying these criteria, a set of 500 SNPs were used to design further PACE assays.

To identify which of the 500 selected SNPs could explain the heritable PM resistance observed in the two F1 populations, systematic testing of the 500 SNPs was performed in a specific order that maximized genome coverage and prioritized lower GWAS p-values. Following this approach, a strong association was observed between the resistance phenotype and a SNP in chromosome 3 of the Cannabis genome (Chromosome ID: NC_044372.1) at ~82 Mbp (SNP: SNC_044372.1_82694448) (FIG. 10). This SNP was designated as MR54. The genomic context sequence of MR54 is provided in Table 5 below with the reference allele underlined and the variant allele as indicated.

TABLE 5

| Marker | SEQ | Position | | |
|---|---|---|---|---|
| (SNP) | ID NO. | in SEQ | Genomic Context Sequence | Variant |
| MR54 | 6 | 76 | TCAAAGTCCAACACKATCGAAAGCTCCACTAATTCTTGACTG | G |
| | | | GGTTCTGYTGTAGAGAGGAAACAKAGCCATGGCTACGTCCAT | |
| | | | KGCCTCTYAATTASAGGCCATCAAWTCCTTCCTACAAGCTAT | |
| | | | TATCTTTAAATCYRAAGCTACTTAA | |

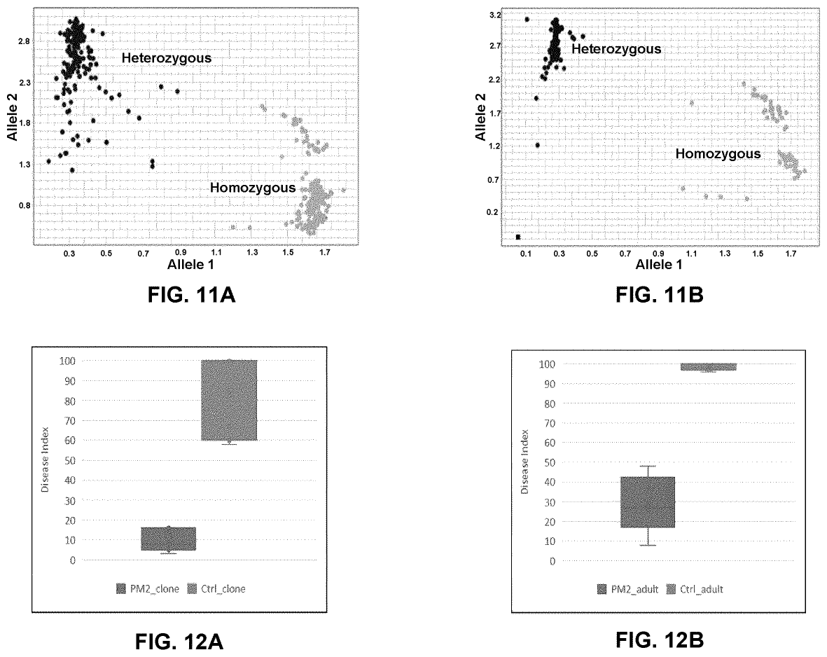

Competitive allele-specific PCR assays (KASP/PACE) were performed to validate the accuracy of the MR54 marker using F1 progenies from crosses between the N88 genotype and a susceptible cultivar from clones (FIG. 11A) and adult plants (FIG. 11B). Genomic DNA samples were extracted from leaf tissue and KASP assays were performed using the KASP genotyping assay standard protocol (LGC Biosearch Technologies™) on a QuantStudio™ 7 qPCR thermocycler (Thermoscientific™). The primers used in the assay are provided in Table 6.

TABLE 6

| | SEQ | |
|---|---|---|
| Primer | ID NO. | Primer Sequence |
| Forward Primer | 7 | GAAGGTGACCAAGTTCATGCTAGAGAGGAAACAKAGCCATGGCT |
| (reference allele) | | |
| Forward Primer | 8 | GAAGGTCGGAGTCAACGGATTGAGAGGAAACAKAGCCATGGCG |
| (variant allele) | | |
| Reverse Primer | 9 | GCTTGTAGGAAGGAWTTGATGGCCT |

The results of the KASP assays are summarized in Table 7 below and show that 136/136 genetically unique individuals with a resistant phenotype were correctly identified as heterozygous and 138/144 unique individuals with a susceptible phenotype were correctly identified as homozygous using the MR54 marker.

TABLE 7

| | | No. of | | No. of | |
|---|---|---|---|---|---|
| | | MR54 | | MR54 | |
| F1 | | marker | | marker | |
| Population | No. of R | heterozygous | No. of S | homozygous | Success |
| tested | scored | hits | scored | hits | rate |
| N88 × AC | 136 | 136 | 144 | 138 | 98% |

The MR54 marker was also tested on several candidate genotypes in the CanD population. Five other genotypes heterozygous for the variant allele of the MR54 marker were detected. Disease index data of these genotypes from both clonal and adult plant assays were compared to the DI of a group of a randomly chosen panel of non-resistant genotypes without the variant allele, which confirmed a strong association between the MR54 marker and PM resistance (FIGS. 12A and 12B).

### Example 7—QTL Mapping using Bulk Segregant Analysis

The initial results from the GWAS analysis discussed above suggested that genotypes containing PM2 resistance were too few within the CanD diversity panel and below the power of detection of GWAS methodologies. To circumvent GWAS limitations, an RNA-based bulk segregant analysis (BSA) approach (“RNA-Seq BSA”) was used on the bi-parental F1 populations made from crosses between PM-resistant and PM-susceptible genotypes (N88×AC and W03×AC).

For each F1 population, susceptible and resistance bulks were created consisting of 25 plants each grouped by their respective phenotype. Equal amounts of leaf samples from the 25 resistant and 25 susceptible plants were collected from PM infected (3 wpi) plants and frozen and the frozen leaf samples were used for RNA isolation.

RNA sequencing and reference genome alignment was performed on the isolated RNA from each bulk. Construction of mRNA libraries and sequencing was performed at SBME-Seq center at the School of Biomedical Engineering, University of British Columbia. Each library was sequenced to a depth of 20 million reads, with 150 bp long reads, using an Illumina NextSeq2000. Library quality and presence of adaptors in raw RNA-Seq reads were analyzed using FastQC (Andrews, 2010). Skewer (Jiang et al., 2014) was used to trim reads to a Phred score no less than Q28. STAR (version 2.7.11a) (Dobin et al., 2013) was used to map processed RNA-Seq reads to either the Pink Pepper or CBDRx reference genomes.

For the W03×AC population, bulks resulted in 25, 150,091 and 17,017,509 reads that were uniquely aligned to the Pink Pepper reference genome for resistant and susceptible bulks, respectively. Similarly, the N88×AC population yielded 24,179,503 and 21,576,849 uniquely mapped reads in the resistant and susceptible bulks, respectively. These reads were used for SNP calling. SNP calling was performed by following The Broad Institute's best practices for RNA-Seq short variant discovery (SNPs+Indels) using GATK (versions 4.3) (McKenna et al., 2010).

A total of 65,202 SNPs from the W03×AC population shared between both bulks were used for BSA after filtering for quality scores and read depth. Similarly, the N88×AC population yielded 82,099 SNPs after filtering that were used for BSA. SNPs called from read counts less than 20 were filtered out as their allelic frequencies cannot be accurately measured.

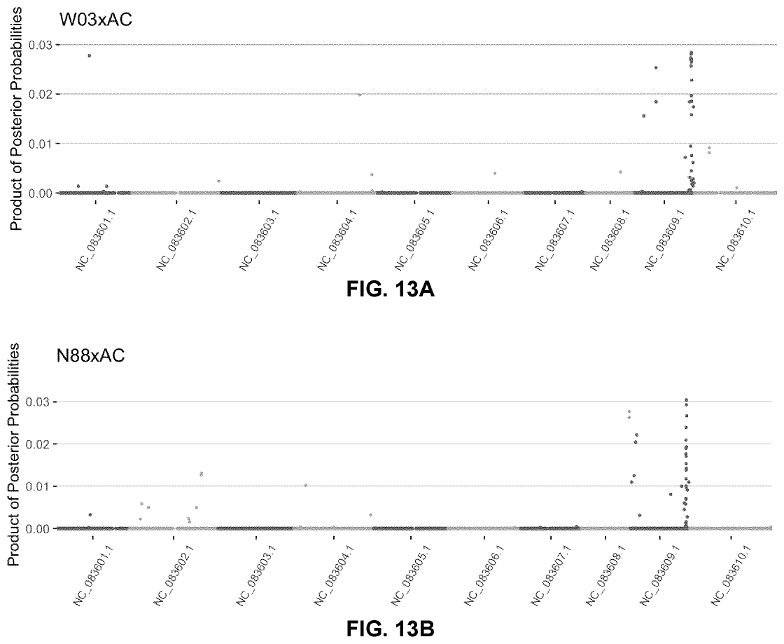

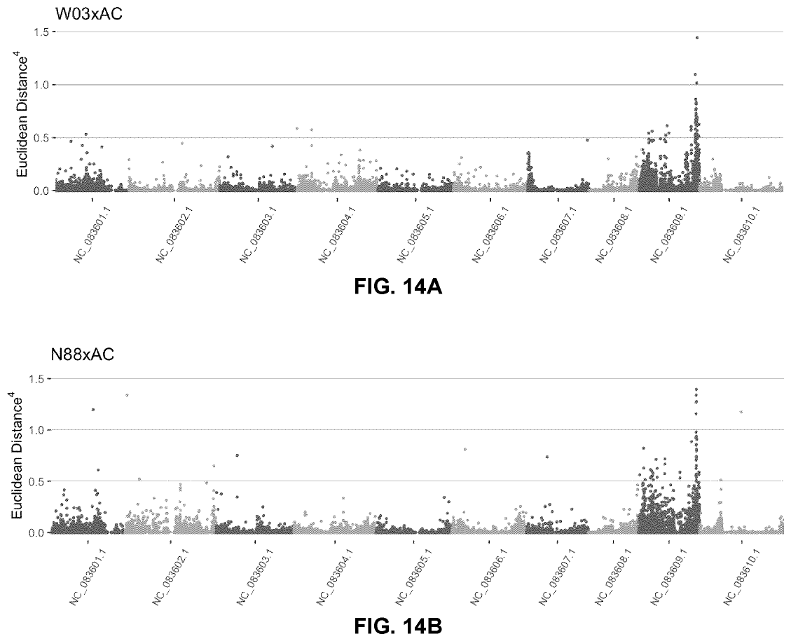

To identify the region associated with PM resistance in these populations, two separate methods were developed for RNA-Seq BSA: the Bayesian implementation by Liu et al. (2012) to estimate the probability of SNPs in linkage disequilibrium with the causal gene, and the Euclidean distance (ED) metric developed by Hill et al. (2013), a robust measure of allele frequency differences between bulks. Both methods rely on allele read counts determined by SNP calling to derive allele frequencies used in their calculations. The Bayesian approach identified a cluster of SNPs with high probability of being linked to PM resistance in chromosomes 9 (NC_083609.1) in both the W03×AC and N88×AC populations (see FIGS. 13A and 13B). Using the Euclidean distance metric, a cluster of SNPs with high ED scores was observed in the same region of chromosome 9 in both populations (see FIGS. 14A and 14B).

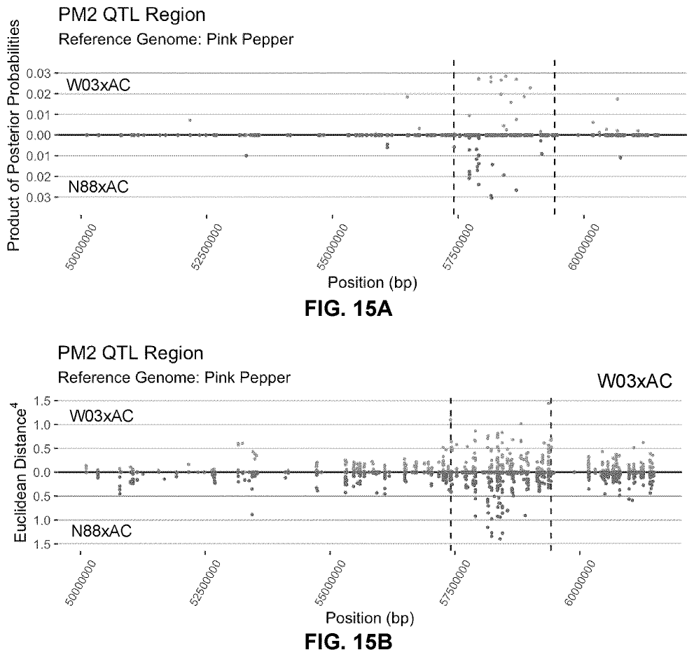

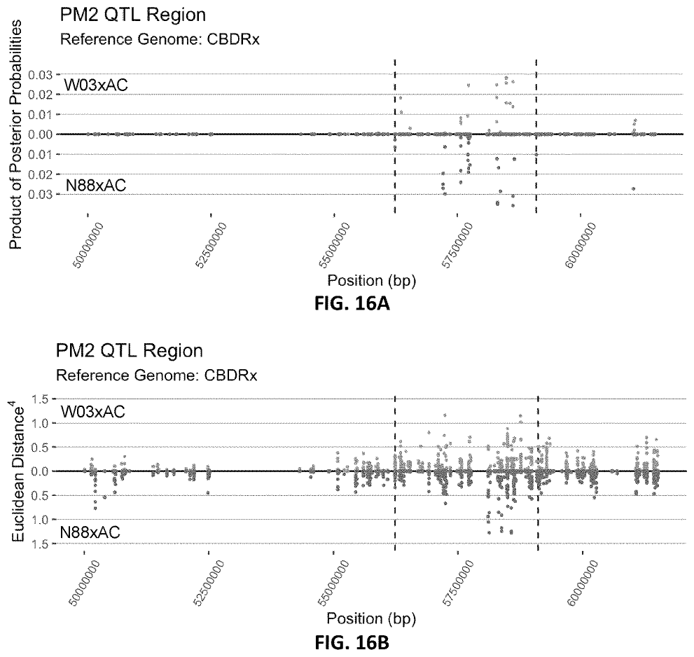

Both the ED and Bayesian probability metrics overlap at an approximately 2.0 Mb region. The boundaries of this region were defined between base pair positions 57,417,178 and 59,418,457 (of the Pink Pepper reference genome) based on SNPs with elevated ED scores and increased posterior probabilities to being linked with PM2 resistance (FIGS. 15A and 15B). To further confirm that the identified region is associated with PM2 resistance, the BSA was repeated on a second publicly available reference genome, CBDRx (cs10). Using both the Bayesian probability and ED metrics, a cluster of SNPs highly associated with PM2 resistance was observed on chromosome 9 (NC_044376.1) corresponding to approximately the same region observed in the Pink Pepper reference genome. This region was defined between base pair positions 56,237,869 and 59,102,577 of chromosome 9 (NC_044376.1) (see FIGS. 16A and 16B).

With reference to the Pink Pepper genome, the PM2 QTL defined region on chromosome 9 contains 2342 and 2208 SNPs for the W03×AC and N88×AC populations, respectively. Each genotype of the CanD diversity population has been previously sequenced with SNPs called using the CBDRx reference genome. Using the defined PM2 QTL boundaries from the BSA in CBDRx, 4,135 SNPs were identified within this region that are heterozygous in both W03 and N88 genotypes and that can potentially be genetic markers to track PM2 resistance.

### Example 8—Identification and Validation of Genetic Markers for PM2 Resistance on Chromosome 9

Five genetic markers for PM2 resistance from the PM2 QTL region on chromosome 9 were identified based on having high ED scores as determined by the BSA results discussed above: MR110, MR121, MR124, MR125, and MR131. The chromosome positions and flanking sequences of these markers are provided in Tables 1 and 2 above. PACE genotyping assays were developed to validate these five markers, as well as the previously identified MR54 marker, on W03×AC and N88×AC F1 populations and W03 and N88 F2 populations.

Genotyping assays were performed on genomic DNA isolated from F1 plants, for example, using a sbeadex Mini Plant DNA Purification Kit (Biosearch Technologies™) on an Oktopure™ Liquid Handling system. Genotyping was done using PCR Allelic Competitive Extension (PACE™) 2.0 SNP genotyping assays (3CR Bioscience™, Essex, UK). Assays were designed by the 3CR Bioscience assay design service. All PACE reactions were performed on a QuantStudio™ 7 (Applied Biosystems™) using the manufacturer-suggested PACE reaction volumes and cycling conditions.

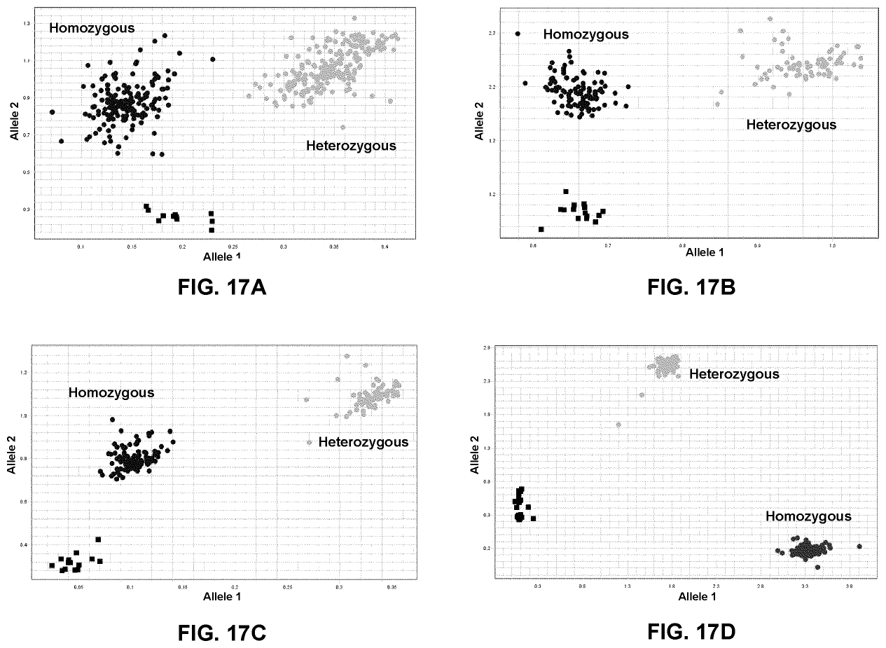

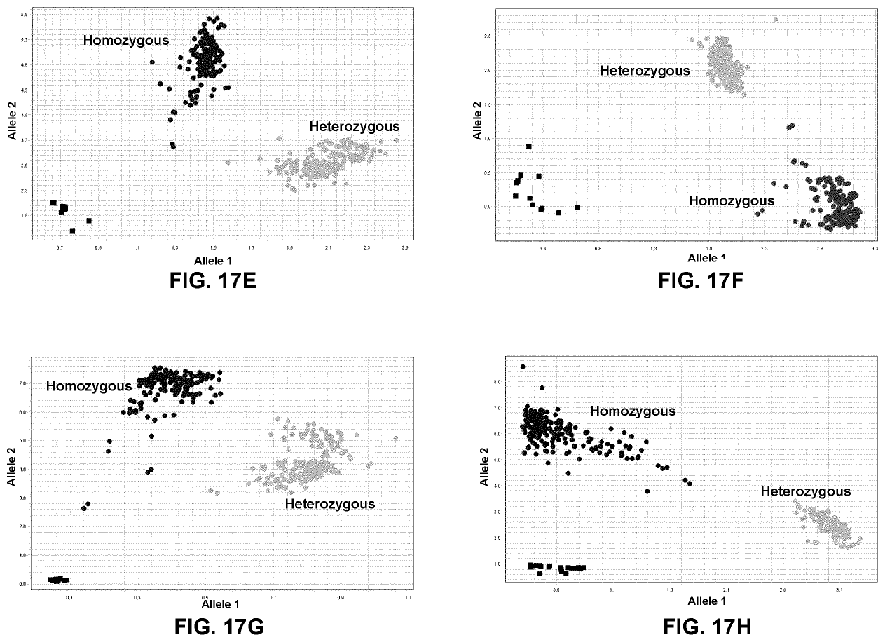

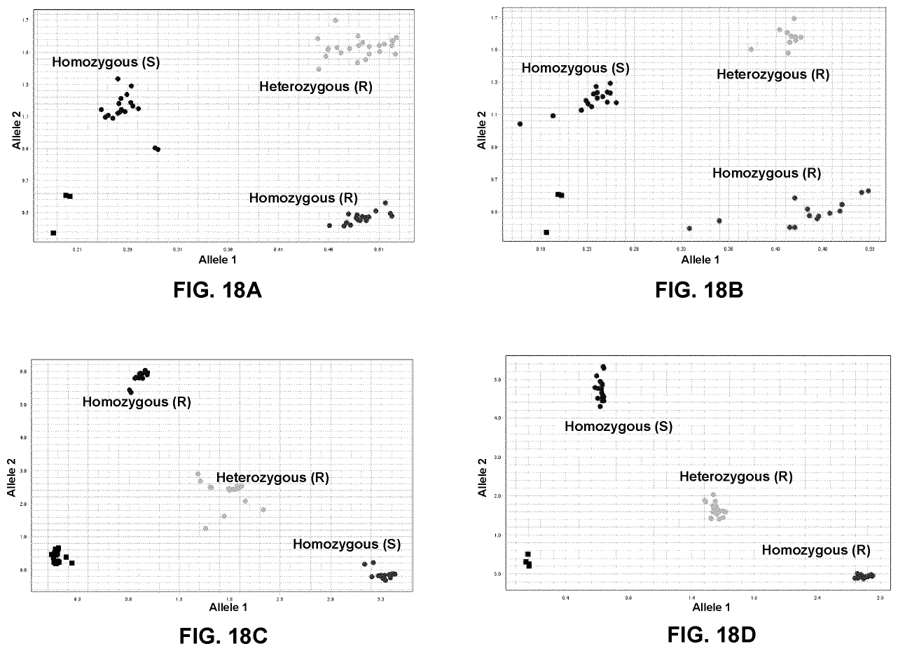

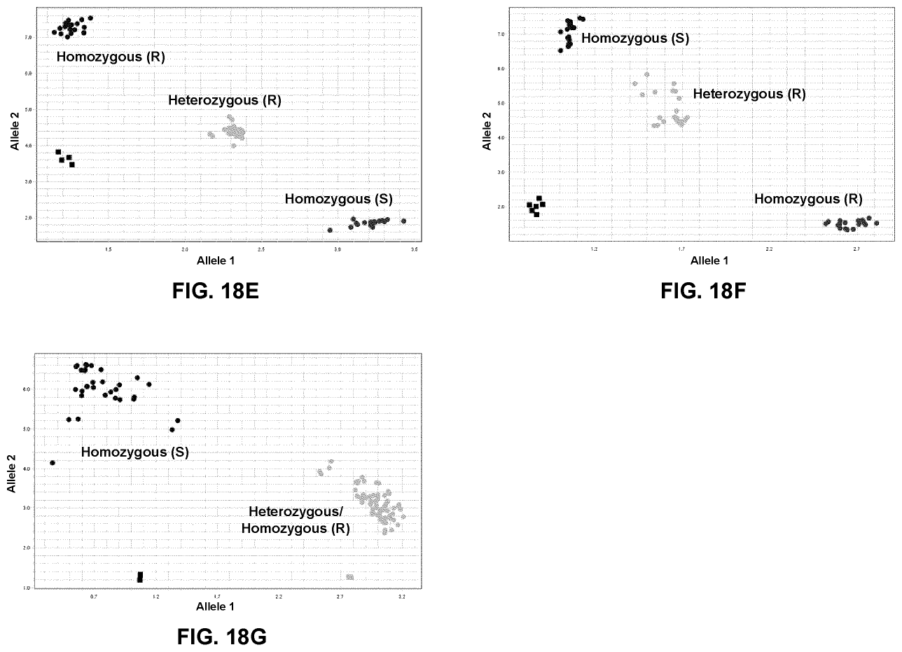

FIGS. 17A-H and 18A-G show the genotyping results for the F1 and F2 populations, respectively. Genotype calls in F2 populations showed 86-93% accuracy when compared to phenotype data from a PM2 clone-infection assay. The marker accuracy is summarized in Table 8 below.

TABLE 8

| Marker | Validated Population | Marker Accuracy | |
|---|---|---|---|
| MR54 | W03 × AC F1 | 92% | (n = 214) |
| | N88 × AC F1 | 98% | (n = 280) |
| | W03 F2 | 100% | (n = 21) |
| | N88 F2 | 93% | (n = 28) |
| MR110 | W03 × AC F1 | 95% | (n = 216)_{—} |
| | N88 × AC F1 | 98% | (n = 187) |
| | W03 F2 | 90% | (n = 21) |
| | N88 F2 | 86% | (n = 28) |
| MR121 | W03 × AC F1 | 92% | (n = 176) |
| | W03 F2 | 90% | (n = 21) |
| MR124 | N88 × AC F1 | 97% | (n = 184) |
| | N88 F2 | 93% | (n = 28) |
| MR125 | N88 × AC F1 | 99% | (n = 180) |
| | N88 F2 | 93% | (n = 28) |
| MR131 | N88 × AC F1 | 99% | (n = 171) |
| | N88 F2 | 93% | (n = 28) |

To evaluate allelic segregation in the F2 populations, a chi-squared test for deviation from Mendelian ratios of inheritance (df=2) was used, and showed all populations fell with accepted ranges (Table 9).

TABLE 9

| | | AA | AA | Aa | Aa | aa | Aa | | p- |
|---|---|---|---|---|---|---|---|---|---|
| Marker | Population | Actual | Expected | Actual | Expected | Actual | Expected | χ² | value |
| MR | N88 F2 | 8 | 7 | 12 | 14 | 8 | 7 | 0.5714 | 0.7515 |
| 110 | | | | | | | | | |
| MR | W03 F2 | 7 | 5.25 | 6 | 10.5 | 8 | 5.25 | 3.9524 | 0.1386 |
| 110 | | | | | | | | | |
| MR | W03 F2 | 8 | 5.25 | 6 | 10.5 | 7 | 5.25 | 3.9524 | 0.1386 |
| 121 | | | | | | | | | |
| MR | N88 F2 | 8 | 7 | 11 | 14 | 9 | 7 | 1.3571 | 0.5074 |
| 124 | | | | | | | | | |
| MR | N88 F2 | 8 | 7 | 12 | 14 | 8 | 7 | 0.5714 | 0.7515 |
| 125 | | | | | | | | | |
| MR | N88 F2 | 8 | 7 | 11 | 14 | 9 | 7 | 1.3571 | 0.5074 |
| 131 | | | | | | | | | |

### Example 9—Mapping of MR54 to Chromosome 9

As discussed above, marker MR54 was initially presumed to be located on chromosome 3 (NC_044372.1) at base pair position 82,694,448 in the CBDRx reference genome, which differs from the other markers located on chromosome 9. To clarify the location of MR54, PCR primers were developed flanking marker MR54 and the resulting amplicon was Sanger sequenced (SEQ ID No. 10).

The full length amplicon (950 bp) was found to align to chromosome 3 (NC_083603.1) of the Pink Pepper genome between base pair position 52,086,220 and 52,085,287 (Bits Score 1618, E-value 0.0). However, a portion of the amplicon between 357 bp and 687 bp had a strong alignment (Bit Score 315, E-value 1.03e-83) to chromosome 9 (NC_083609.1) between base pair position 58,199,370 and 58,199,055, a region within the defined PM2 QTL and between markers MR124 and MR125. Furthermore, the forward PACE primer from MR54 was found to align to this homologous region. Given the association of marker MR54 with PM2 resistance, marker MR54 is likely located in chromosome 9 and its initial position in chromosome 3 may be due to an assembly error in the Cannabis reference genomes.

PCR amplicons containing markers 110 and 131 were also Sanger sequenced (SEQ ID NO. 11 and SEQ ID NO. 12, respectively) and aligned to the Pink Pepper reference genome to confirm their location. Both marker positions were at the positions on chromosome 9 as expected.

### Example 10—Additional Genetic Marker Candidates

Additional genetic markers were identified from the PM2 QTL region on the Pink Pepper genome by filtering the variants down to ones that: 1) are present in one bulk but not the other (resistant vs. susceptible); 2) are found in both populations (N88×AC and W03×AC); and 3) have reasonable read coverage for variant identification. Genetic markers meeting these criteria in which the variant was present in the resistant bulk but not the susceptible bulk are listed in Table 3 above.

Several genetic markers were also identified in which the variant was present in the susceptible bulk and not the resistant bulk. These markers are listed in Table 10 below and may be useful in breeding techniques to select against a PM susceptible phenotype.

TABLE 10

| | Type of | |
|---|---|---|
| Position | Polymorphism | Variant |
| 58153797 | SNP | C > T |
| 58245609 | SNP | G > A |
| 58245611 | SNP | G > A |
| 58245616 | SNP | A > C |
| 58245620 | SNP | A > G |
| 58245623 | SNP | G > A |
| 58245624 | SNP | C > T |
| 58245630 | SNP | T > C |
| 58938679 | Duplication | dupCAA |
| 58938900 | SNP | T > C |
| 59224361 | SNP | G > T |

The positions of each polymorphic site are with reference to chromosome 9 of the Pink Pepper genome (NC_083609.1). For SNPs, the Reference>Variant nucleotides are shown. For the duplication, the duplicated (dup) sequence is shown.

Although a few embodiments have been shown and described, it will be appreciated by those skilled in the art that various changes and modifications can be made to those skilled in the art that various changes and modifications can be made to these embodiments without changing or departing from their scope, intent or functionality. The terms and expressions used in the preceding specification have been used herein as terms of description and not of limitation, and there is no intention in the use of such terms and expressions of excluding equivalents of the features shown and described or portions thereof.

### REFERENCES

The following documents are incorporated by reference herein:
AGRIOS G N (2005) PLANT DISEASES CAUSED BY FUNGI. Plant Pathol. Elsevier, pp 385-614
Andrews, S. (2010). FASTQC. A quality control tool for high throughput sequence data
Dobin, A., Davis, C. A., Schlesinger, F., Drenkow, J., Zaleski, C., Jha, S., . . . & Gingeras, T. R. (2013). STAR: ultrafast universal RNA-seq aligner. Bioinformatics, 29 (1), 15-21.
Hill, J. T., Demarest, B. L., Bisgrove, B. W., Gorsi, B., Su, Y. C., & Yost, H. J. (2013). MMAPPR: mutation mapping analysis pipeline for pooled RNA-seq. Genome research, 23 (4), 687-697.
Hückelhoven R (2005) Powdery mildew susceptibility and biotrophic infection strategies. FEMS Microbiol Lett 245:9-17
Jiang, H., Lei, R., Ding, S. W., & Zhu, S. (2014). Skewer: a fast and accurate adapter trimmer for next-generation sequencing paired-end reads. BMC bioinformatics, 15, 1-12.
Liu, S., Yeh, C. T., Tang, H. M., Nettleton, D., & Schnable, P. S. (2012). Gene mapping via bulked segregant RNA-Seq (BSR-Seq). PloS one, 7 (5), e36406.
Mihalyov PD, Garfinkel AR (2021) Discovery and Genetic Mapping of PM1, a Powdery Mildew Resistance Gene in Cannabis sativa L. Frontiers in Agronomy. doi: 10.3389/fagro.2021.720215
McKenna, A., Hanna, M., Banks, E., Sivachenko, A., Cibulskis, K., Kernytsky, A., . . . & DePristo, M. A. (2010). The Genome Analysis Toolkit: a MapReduce framework for analyzing next-generation DNA sequencing data. Genome research, 20 (9), 1297-1303.
Pépin N, Punja Z K, Joly D L (2018) Occurrence of Powdery Mildew Caused by Golovinomyces cichoracearum sensu lato on Cannabis sativa in Canada. Plant Dis 102:2644-2644
Scott C, Punja Z K (2021) Evaluation of disease management approaches for powdery mildew on Cannabis sativa L. (marijuana) plants. Canadian Journal of Plant Pathology 43:394-412
Seifi A, Gao D, Zheng Z, Pavan S, Faino L, Visser RGF, Wolters A-MA, Bai Y (2014) Genetics and molecular mechanisms of resistance to powdery mildews in tomato (Solanum lycopersicum) and its wild relatives. Eur J Plant Pathol 138:641-665

Seifi H (Soren), Serajazari M, Kaviani M, Pauls P, Booker H, Navabi A (2021) Immunity to stripe rust in wheat: A case study of a hypersensitive-response (HR)-independent resistance to Puccinia striiformis f. sp. tritici in Avocet-Yr15. Canadian Journal of Plant Pathology 43: S188-S197

Seifi H S, Curvers K, Vleesschauwer D, Delaere I, Aziz A, Höfte M (2013) Concurrent overactivation of the cytosolic glutamine synthetase and the GABA shunt in the ABA-deficient sitiens mutant of tomato leads to resistance against Botrytis cinerea. New Phytologist 199:490-504

1. A method comprising:
providing nucleic acid from a Cannabis plant; and
testing the nucleic acid to determine the presence or absence of a variant at a polymorphic site on chromosome 9 of the Cannabis genome, wherein the presence of the variant in one or both copies of the polymorphic site indicates that the plant has a powdery mildew resistant phenotype.

2. The method of claim 1, wherein the polymorphic site is located within a region on chromosome 9 of the Pink Pepper reference genome selected from: between about 57 megabases to about 60 megabases, between about 57.4 to about 59.5 megabases, between about 58 to about 59 megabases, between about 58.1 to about 58.6 megabases, or between about 58,164,118 and about 58,552,299 bases.

3. The method of claim 1, wherein the polymorphic site is located within a region on chromosome 9 of the CBDRx reference genome selected from: between about 56 megabases to about 60 megabases, between about 56.2 to about 59.2 megabases, between about 58 to about 59 megabases, between about 58.1 to about 58.6 megabases, or between about 58,108,801 to about 58,570,898 bases.

4. The method of claim 1, wherein the polymorphic site is selected from: position 101 of SEQ ID NO: 1 or its complement; position 101 of SEQ ID NO: 2 or its complement; position 101 of SEQ ID NO: 3 or its complement; position 101 of SEQ ID NO: 4 or its complement; position 101 of SEQ ID NO: 5 or its complement; or another polymorphic site in linkage disequilibrium therewith.

5. The method of claim 4, wherein:
the polymorphic site is position 101 of SEQ ID NO: 1 or its complement, and wherein the variant is an A or a T in the complement;
the polymorphic site is position 101 of SEQ ID NO: 2 or its complement, and wherein the variant is an A or a T in the complement;
the polymorphic site is position 101 of SEQ ID NO: 3 or its complement, and wherein the variant is a G or a C in the complement;
the polymorphic site is position 101 of SEQ ID NO: 4 or its complement, and wherein the variant is a T or an A in the complement; or
the polymorphic site is position 101 of SEQ ID NO: 5 or its complement, and wherein the variant is a T or an A in the complement.

6. The method of claim 1, wherein the polymorphic site is one of the polymorphic sites in Table 3 or another polymorphic site in linkage disequilibrium therewith.

7. The method of claim 1, wherein the nucleic acid is genomic DNA.

8. The method of claim 1, wherein the testing comprises nucleic acid amplification.

9. The method of claim 1, wherein the testing is performed using an allele specific method.

10. The method of claim 1, wherein the testing is performed using allele-specification amplification, allele-specific probe hybridization, allele-specific primer extension, sequencing, 5′ nuclease digestion, molecular beacon assay, oligonucleotide ligation assay, size analysis, single-stranded conformation polymorphism analysis, denaturing gradient gel electrophoresis (DGGE), or a combination thereof.

11. A method for producing a Cannabis plant with a powdery mildew resistant phenotype, comprising:
identifying a first parent plant having a variant at a polymorphic site on chromosome 9 of the Cannabis genome, wherein the presence of the variant in one or both copies of the polymorphic site indicates that the plant has a powdery mildew resistant phenotype;
crossing the first parent plant with a second parent plant to product F1 progeny; and
identifying a first F1 progeny plant that has the variant at the polymorphic site and/or that has the powdery mildew resistant phenotype.

12. The method of claim 11, wherein at least one of the first parent plant and the first F1 progeny plant are identified as having the variant using the method of any one of claims 1 to 10.

13. The method of claim 11, further comprising:
crossing the first F1 progeny plant to a second F1 progeny plant to produce F2 progeny; and
identifying a first F2 progeny plant that has the variant and/or that has the powdery mildew resistant phenotype.

14. The method of claim 11, further comprising:
self-pollinating the first F1 progeny plant to produce F2 progeny; and
identifying a first F2 progeny plant that has the variant at the polymorphic site and/or that has the powdery mildew resistant phenotype.

15. The method of claim 11 or 12, further comprising:
backcrossing the first F1 progeny plant with the first parent plant or the second parent plant to produce F2 progeny; and
identifying a first F2 progeny plant that has the variant and/or that has the powdery mildew resistant phenotype.

16. A method for producing a plant with a powdery mildew resistant phenotype, comprising:
identifying a first parent plant having a variant at a polymorphic sit on chromosome 9 of the Cannabis genome, wherein the presence of the variant in one or both copies of the polymorphic site indicates that the plant has a powdery mildew resistant phenotype;
crossing the first parent plant with a second parent plant to product F1 progeny;
crossing a first F1 progeny plant to produce F2 progeny, wherein the first F1 progeny plant is crossed to one of: a second F1 progeny plant, the first parent plant, the second parent plant, and itself; and
identifying a first F2 progeny plant that has the variant and/or that has the powdery mildew resistant phenotype.

17. The method of claim 16, wherein at least one of the first parent plant and the first F2 progeny plant are identified as having the variant using the method of any one of claims 1 to 10.

18. A method for producing a Cannabis plant, comprising crossing a Cannabis plant produced according to the method of claim 11 to a second Cannabis plant having at least one other desired trait.

19. The method of claim 18, wherein the at least one other desired trait comprises a different powdery mildew resistance phenotype.

20. A Cannabis plant or a plant cell produced according to the method of claim 11.
